SEQUENCE LISTING (5 sequences)

SEQ ID NO: 1 - protein, 356 aa, Homo sapiens
MSADAAAGAPLPRLCCLEKGPNGYGFHLHGEKGKLGQYIRLVEPGSPAEKAGLLAGDRLVEVNGENVEKETHQQWSRIRAALNAVRLLVVDPETDEQLQKLGVQVREELLRAQEAPGQAEPPAAAEVQGAGNENEPREADKSHPEQRELRPRLCTMKKGPSGYGFNLHSDKSKPGQFIRSVDPDSPAEASGLRAQDRIVEVNGVCMEGKQHGDWSAIRAGGDETKLLVVDRETDEFFKKCRVIPSQEHLNGPLPVPFTNGEIQKENSREALAEAALESPRPALVRSASSDTSEELNSQDSPPKQDSTAPSSTSSSDPILDFNISLAMAKERAHQKRSSKRAPQMDWSKKNELFSNL

SEQ ID NO: 2 - DNA, 42 bp, Artificial Sequence
  Description of Artificial Sequence Synthetic primer
atgcatcatcaccatcaccatcacagcgcggacgcagcggcc

SEQ ID NO: 3 - DNA, 47 bp, Artificial Sequence
  Description of Artificial Sequence Synthetic primer
cgggctttgtttagcagcctagttatcagaggttgctgaagagttcg

SEQ ID NO: 4 - protein, 7 aa, Artificial Sequence
  Description of Artificial Sequence Synthetic 7xHis tag
HHHHHHH

SEQ ID NO: 5 - DNA, 45 bp, Artificial Sequence
  Description of Artificial Sequence Synthetic primer
ctttaagaaggagatatacatatgcatcatcaccatcaccatcac

(19) United States
(12) Patent Application Publication
(10) Pub. No.: US 2010/0272635 A1
(43) Pub. Date: Oct. 28, 2010

(54) METHODS AND COMPOSITIONS FOR DIAGNOSIS AND/OR PROGNOSIS IN OVARIAN CANCER AND LUNG CANCER

(76) Inventors: Kelline M. Rodems, Oceanside, CA (US); David W. Oelschlager, San Diego, CA (US); Uday Kumar Veeramallu, San Diego, CA (US); Joseph Buechler, Carlsbad, CA (US)

Correspondence Address: WILSON, SONSINI, GOODRICH & ROSATI, 650 PAGE MILL ROAD, PALO ALTO, CA, 94304-1050, US

(21) Appl. No.: 12/663,699

(22) PCT Filed: Jun. 13, 2008
(86) PCT No.: PCT/US08/66961

§ 371 Date: Jul. 6, 2010

Related U.S. Application Data
(60) Provisional application No. 60/934,737, filed on Jun. 15, 2007.
(60) Provisional application No. 60/934,735, filed on Jun. 15, 2007.

(51) Int. Cl. A61K 51/00; A61K 49/00; A61K 49/06; A61K 49/22; G01N 33/53

(52) U.S. Cl. 424/1.11; 424/9.1; 424/9.3; 424/9.5; 435/7.94

(57) ABSTRACT

Methods and compositions for diagnosis, prognosis and monitoring of ovarian cancer and lung cancer are provided. Assays that detect NHERF-I (or one or more markers related thereto) and NHERF-I-containing complexes are used to assign a diagnosis to a subject being assessed for the presence of ovarian or lung cancer; assign a prognostic risk to a subject suffering from ovarian or lung cancer; or monitor the course of ovarian or lung cancer treatment in a subject.

### CROSS-REFERENCE TO RELATED APPLICATIONS

This application claims the benefit of U.S. Provisional Application No. 60/934,735, filed Jun. 15, 2007, and U.S. Provisional Application No. 60/934,737, filed Jun. 15, 2007, both incorporated herein by reference.

### FIELD OF THE INVENTION

The present invention relates to the identification and use of diagnostic markers related to cancer.

### BACKGROUND OF THE INVENTION

The following discussion of the background of the invention is merely provided to aid the reader in understanding the invention and is not admitted to describe or constitute prior art to the present invention.

Ovarian Cancer

Ovarian cancer is the fourth leading cause of cancer-related deaths in women in the United States, with a lifetime risk of about 1 in 70 women, and about 1 in 100 women dying of the disease. Most ovarian cancers happen in postmenopausal women, with half of all ovarian cancers found in women over the age of 63. A history of colorectal, endometrial or breast cancer, either personally or in immediate relatives, are risk factors for ovarian cancer. Additional risk factors include obesity, the use of clomiphene (as an infertility treatment), early onset of menses, late menopause, women who are nulliparous, women having late primagravida, use of estrogen replacement therapies, smoking, and alcohol use.

Ovarian cancers are often based on the tissue type of origin. There are three main types of tumors: germ cell tumors, stromal tumors, and epithelial tumors. Ovarian cancers of epithelial origin may be further divided into Tumors of low malignant potential (LMP tumors or borderline tumors) and epithelial ovarian cancers. Nearly 9 out of 10 ovarian cancers are of the latter type, while only about 1 in 20 ovarian cancers are germ cell tumors.

Ovarian cancers are often staged surgically. “Staging” refers to a process by which the invasiveness and aggressiveness of a tumor is assessed. The AJCC/TNM system may be used to stage the cancer. This system describes the cancer in terms of the extent of the tumor (T), whether or not it has spread to nearby lymph nodes (N), and whether it has spread to organs farther away, or metastasized (M).

Stage I refers to ovarian cancer that is contained within the ovary (or ovaries); Stage II refers to ovarian cancer that is in one or both ovaries and has spread to other organs in the pelvis, such as the bladder, colon, rectum, or uterus; Stage III refers to ovarian cancer that is in one or both ovaries and has spread to the lining of the abdomen or to the lymph nodes; and Stage IV refers to ovarian cancer that has spread from one (or both) ovaries to distant organs, such as the liver or lungs, or for example, cancer cells in the fluid around the lungs. Stages I and II are sometimes further divided into IA (tumor limited to one ovary; no tumor on the external surface, and capsule intact); IB (tumor limited to both ovaries; no tumor on the external surface, and capsules intact); IC (stage IA or IB but with tumor on the surface of one or both ovaries, with capsule ruptured, or with ascites or peritoneal washings containing malignant cells); IIA (extension and/or metastases to the uterus, fallopian tubes, or both); IIB (extension to other pelvic tissues); and IIC (stage IIA or IIB but with tumor on the surface of one or both ovaries, with capsule ruptured, or with ascites or peritoneal washings containing malignant cells).

The 5-yr survival rates with treatment are 70 to 100% with stage I, 50 to 70% with stage II, 15 to 35% with stage III, and 10 to 20% with stage IV. Prognosis is worse when tumor grade is higher or when surgery cannot remove all visibly involved tissue; then, prognosis is best when the involved tissue can be reduced to <1 cm in diameter. With stages III and IV, recurrence rate is about 70%. Unfortunately, the signs and symptoms of ovarian cancer are often nonspecific, and about 75% of ovarian cancer cases present with advanced stage disease.

Lung Cancer

Lung cancer is second only to ischemic heart disease as the most frequent cause of death in the United States. The lifetime risk of developing lung cancer for males who have never smoked is 1 in 76, for past smokers 1 in 12, and for current smokers 1 in 4.5. For females who have never smoked it is 1 in 157, for past smokers 1 in 23, and for current smokers 1 in 8.8. From the time of diagnosis, approximately six out often people with lung cancer die in the first year, between seven and eight in ten die within 2 years, and only about 11 to 15 percent of those afflicted will live beyond five years.

It has been estimated that 90% of lung cancer cases in men and 80% in women are the result of exposure to tobacco smoke. Other risk factors include age, radon exposure, asbestos exposure, family history of lung cancer, and chronic pulmonary inflammation such as that caused by tuberculosis or other scarring diseases.

Bronchogenic carcinomas, which account for more than 90% of all lung cancers, can be subdivided into four major histologic types: squamous cell carcinoma, adenocarcinoma, large cell carcinoma, and small cell carcinoma. Often, two or more of these histologic types occur together in the same patient. Squamous cell carcinoma accounts for about 50% of lung cancers among patients older than 65 years. Adenocarcinoma accounts for another 30 to 35% of lung cancers in this elderly population. Except for stage I lesions, adenocarcinoma generally has a worse prognosis than squamous cell carcinoma. Large cell carcinoma accounts for 15% of all lung cancers. Small cell (oat cell) carcinoma accounts for 15 to 20% of all lung cancers, though it is somewhat more common in patients over 65 (accounting for slightly more than 25% of lung cancers in this population). Small cell carcinoma is the most rapidly growing and most responsive to chemotherapy of all lung cancers.

Lung cancers are often staged surgically. The following summarizes the staging of lung cancers:

| Overall Stage | T Stage | N Stage | M Stage |
|---|---|---|---|
| Stage 0 | Tis (In situ) | N0 | M0 |
| Stage IA | T1 | N0 | M0 |
| Stage IB | T2 | N0 | M0 |
| Stage IIA | T1 | N1 | M0 |
| Stage IIB | T2 | N1 | M0 |
| | T3 | N0 | M0 |
| Stage IIIA | T1 | N2 | M0 |
| | T2 | N2 | M0 |
| | T3 | N1 | M0 |
| | T3 | N2 | M0 |
| Stage IIIB | AnyT | N3 | M0 |
| | T4 | AnyN | M0 |
| Stage IV | AnyT | AnyN | M1 |

TX: Primary tumor cannot be assessed, or tumor proven by the presence of malignant cells in sputum or bronchial washings but not visualized by imaging or bronchoscopy
T0: No evidence of primary tumor
Tis: Carcinoma in situ
T1: Tumor 3 cm or less in greatest dimension surrounded by lung or visceral pleura, without bronchoscopic evidence of invasion more proximal than the lobar bronchus (not in the main bronchus)
T2: Tumor with any of the following features of size or extent: more than 3 cm in greatest dimension; involves main bronchus, 2 cm or more distal to the carina; invades the visceral pleura; or associated with atelectasis or obstructive pneumonitis that extends to the hilar region but does not involve the entire lung
T3: Tumor of any size that directly invades any of the following: chest wall (including superior sulcus tumors), diaphragm, mediastinal pleura, parietal pericardium; or tumor in the main bronchus less than 2 cm distal to the carina, but without involvement of the carina; or associated atelectasis or obstructive pneumonitis of the entire lung
T4: Tumor of any size that invades any of the following: mediastinum, heart, great vessels, trachea, esophagus, vertebral body, carina; or separate tumor nodules in the same lobe; or tumor with a malignant pleural effusion.
NX: Regional lymph nodes cannot be assessed
N0: No regional lymph node metastasis
N1: Metastasis to ipsilateral peribronchial and/or ipsilateral hilar lymph nodes and intrapulmonary nodes including involvement by direct extension of the primary tumor
N2: Metastasis to ipsilateral mediastinal and/or subcarinal lymph node(s)
N3: Metastasis to contralateral mediastinal, contralateral hilar, ipsilateral or contralateral scalene, or supraclavicular lymph node(s)
MX: Distant metastasis cannot be assessed
M0: No distant metastasis
M1: Distant metastasis (includes synchronous separate nodule(s) in a different lobe).

Lung cancer may initially be asymptomatic, or accompanied by rather nonspecific symptoms. The most common initial symptom of lung cancer is a persistent cough. Because many of the causes of lung cancer also result in a persistent cough, those who develop lung cancer often present for evaluation noting that their coughing has become worse. If sputum is present, it may be streaked with blood (called “hemoptysis”). Lung cancer may also cause wheezing by narrowing the bronchus in or around which it is growing, which may ultimately lead to the collapse of the part of the lung that the bronchus supplies (called “atelectasis”). Other consequences of a blocked bronchus are shortness of breath, pneumonia, fever, and chest pain.

As the tumor mass becomes greater, it may grow into nerves in the neck, causing a droopy eyelid, small pupil, sunken eye, and reduced perspiration on one side of the face, all symptoms of Homer's syndrome. Cancers at the top of the lung may also grow into the nerves that supply the arm, making the arm painful, numb, and weak, symptoms of Pancoast syndrome. The tumor may also grow into the esophagus, causing difficulty in swallowing; or into the heart and its associated vessels, causing abnormal cardiac rhythms, blockage of cardiac blood flow, pericardial fluid accumulation, and compression of the superior vena cava.

On average, people with untreated lung cancer survive 8 months. For patients with adequate pulmonary reserve and no evidence of distant metastases, the extent of mediastinal and thoracic lymph node involvement determines whether curative surgical resection is possible. For patients that are poor surgical candidates, initial staging may be performed via mediastinoscopic or needle transtracheal lymph node sampling. If the tumor is found to extend to the mediastinum, the disease is considered incurable and surgery is not beneficial. Curative surgical treatment for non-small cell lung cancer may be considered for patients with stage I or II disease, but is often not useful for patients with advanced (stage IIIB, stage IV) non-small cell lung cancer. Cure with chemotherapy is rare. But in the case of advanced-stage small cell lung cancer, extension of lifespan can be realized (raising the 2-year survival rate to 10%), making chemotherapy an appropriate option. Radiation can be used as primary therapy or to help ameliorate symptoms in advanced disease. Patients with moderately advanced (T3, T4, N2, or N3) non-small cell cancer often receive radiation therapy alone or combined with chemotherapy as primary treatment. Patients with distant metastases (Ml) receive radiation therapy for palliation and pain control. When staging studies confirm that small cell cancer is limited, reported median survival is 10 to 16 months with radiation therapy.

The spread of lung cancer may occur early in the disease, especially in the case of small cell carcinoma, often before any lung problems become evident, making an early diagnosis difficult. Overall, even with therapy, the 5-year survival rate is only 13%. Because small cell carcinoma has almost always spread beyond the lung at the time of diagnosis, its prognosis is generally worse than for other types of lung cancer.

### SUMMARY OF THE INVENTION

### Ovarian Cancer

In some aspects, the disclosure provides methods relating to the identification and use of markers for the diagnosis of ovarian cancer, for stratification of risk in ovarian cancer patients, and for monitoring therapy in ovarian cancer patients. The methods and compositions provided herein can be used to facilitate the treatment of patients and the development of additional diagnostic and/or prognostic indicators and therapies. Various aspects relate to materials and procedures for measuring Na+/H+ exchange regulatory cofactor NHE-RF (hereinafter “NHERF-1”) and/or one or more related markers, for the use of NHERF-1 and/or its related markers as a diagnostic marker in ovarian cancer, for the use of NHERF-1 and/or its related markers in treating a patient and/or to monitor the course of a treatment regimen; and for the use of NHERF-1 and/or its related markers to identify subjects at risk for one or more adverse outcomes related to ovarian cancer.

In one aspect, methods of assigning a diagnosis to a subject being assessed for the presence or absence of ovarian cancer are provided, the methods comprising performing an assay that detects NHERF-1 or a marker related thereto on a sample obtained from the subject to provide an assay result, and relating the assay result to the presence or absence of ovarian cancer in the subject.

In yet another aspect, methods of assigning a prognostic risk to a subject diagnosed with ovarian cancer are provided, the methods comprising performing an assay that detects NHERF-1 or a marker related thereto on a sample obtained from the subject to provide an assay result, and relating the assay result to the likelihood of an outcome related to ovarian cancer in the subject.

In still another aspect, methods of monitoring a treatment regimen in a subject being treated for ovarian cancer are provided, the methods comprising performing an assay that detects NHERF-1 or a marker related thereto on a sample obtained from the subject to provide an assay result, and relating the assay result to the success or failure of the treatment received by the subject.

In still another aspect, methods of assigning a diagnosis to a subject being assessed for the presence of ovarian cancer, assigning a prognostic risk to a subject suffering from ovarian cancer, and/or monitoring the course of ovarian cancer treatment in a subject are provided. The methods comprise performing an assay that detects one or more markers of a NHERF-1-containing complex on a sample obtained from the subject to provide an assay result. The methods may further comprise relating the assay result obtained to the presence or absence of ovarian cancer in the subject, to the likelihood of an outcome related to ovarian cancer in the subject, and/or to the success or failure of treatment for ovarian cancer received by the subject. In some embodiments, the NHERF-1-containing complex comprises NHERF-1 and podocalyxin-like protein 1.

Typically, relating one or more assay results to a particular clinical endpoint of interest (such as, e.g., the presence or absence of ovarian cancer, a prognostic risk, or the relative success of a treatment) comprises comparing an individual assay result to a threshold value. For markers that increase as a result of the clinical endpoint, such as NHERF-1, a test value obtained from the subject under study that is greater than the threshold value assigns an increased risk of disease relative to a risk assigned when the value is less than the threshold value, and/or a test value obtained from the subject under study that is less than the threshold value assigns a decreased risk of disease relative to a risk assigned when the value is greater than the threshold value.

Lung Cancer

In some aspects, the disclosure provides methods relating to the identification and use of markers for the diagnosis of lung cancer, for stratification of risk in lung cancer patients, and for monitoring therapy in lung cancer patients. Methods and compositions disclosed herein can be used to facilitate the treatment of patients and the development of additional diagnostic and/or prognostic indicators and therapies.

Various aspects relating to materials and procedures for measuring Na+/H+ exchange regulatory cofactor NHE-RF (hereinafter “NHERF-1”) and/or one or more related markers, for the use of NHERF-1 and/or its related markers as a diagnostic marker in lung cancer, for the use of NHERF-1 and/or its related markers in treating a patient and/or to monitor the course of a treatment regimen; and for the use of NHERF-1 and/or its related markers to identify subjects at risk for one or more adverse outcomes related to lung cancer are provided.

In one aspect, methods of assigning a diagnosis to a subject being assessed for the presence or absence of lung cancer are provided, the methods comprising performing an assay that detects NHERF-1 or a marker related thereto on a sample obtained from the subject to provide an assay result, and relating the assay result to the presence or absence of lung cancer in the subject.

In another aspect, methods of assigning a prognostic risk to a subject diagnosed with lung cancer are provided comprising, performing an assay that detects NHERF-1 or a marker related thereto on a sample obtained from the subject to provide an assay result, and relating the assay result to the likelihood of an outcome related to lung cancer in the subject.

In yet another aspect, methods of monitoring a treatment regimen in a subject being treated for lung cancer are provided, the methods comprising performing an assay that detects NHERF-1 or a marker related thereto on a sample obtained from the subject to provide an assay result, and relating the assay result to the success or failure of the treatment received by the subject.

In still another aspect, methods of assigning a diagnosis to a subject being assessed for the presence of lung cancer, assigning a prognostic risk to a subject suffering from lung cancer, and/or monitoring the course of lung cancer treatment in a subject are provided. The methods comprise performing an assay that detects one or more markers of a NHERF-1-containing complex on a sample obtained from a subject to provide an assay result. The methods may further comprise relating the assay result obtained to the presence or absence of lung cancer in a subject, to the likelihood of an outcome related to lung cancer in the subject, and/or to the success or failure of treatment for lung cancer received by the subject. In some embodiments, the NHERF-1-containing complex comprises NHERF-1 and podocalyxin-like protein 1.

Relating one or more assay results to a particular clinical endpoint of interest (e.g., the presence or absence of lung cancer, a prognostic risk, or the relative success of a treatment) comprises comparing an individual assay result to a threshold value. For markers that increase as a result of the clinical endpoint such as NHERF-1, a test value obtained from the subject under study that is greater than the threshold value assigns an increased risk of disease relative to a risk assigned when the value is less than the threshold value, and/or a test value obtained from the subject under study that is less than the threshold value assigns a decreased risk of disease relative to a risk assigned when the value is greater than the threshold value.

Lung and Ovarian Cancer

In certain embodiments of all of the aspects described herein, NHERF-1 may be combined with additional markers. Numerous methods for combining diagnostic markers are known in the art. These methods typically comprise comparing each marker to a respective threshold value. However, methods in which multiple assay results are combined into a single composite value are known in the art. In such methods, the composite result is typically compared to a threshold value, rather than the individual assay results. Markers that may be useful in combination with NHERF-1 for ovarian cancer applications include, but are not limited to, podocalyxin-like protein 1, the β subunit of human chorionic gonadotropin ((β-hCG), lactate dehydrogenase (LDH), α-fetoprotein, inhibin, osteopontin, human epididymis protein 4 (HE4, WFDC2), and cancer antigen 125 (CA 125). Other suitable markers are described hereinafter. In addition, NHERF-1, alone or with these other markers, may also be combined with the results of additional medical studies such as ultrasonography, transvaginal Doppler flow studies, computed tomography (CT), magnetic resonance imaging (MRI), and biopsy to arrive at a diagnosis, prognosis, or treatment result.

The skilled artisan will understand that numerous methods may be used to select a threshold value for a particular marker or a plurality of markers. In diagnostic aspects, a threshold value may be obtained by performing the assay method on samples obtained from a population of patients having a certain type of cancer, and from a second population of subjects that do not have cancer. For prognostic or treatment monitoring applications, a population of patients, all of which have, for example, ovarian cancer, may be followed for the time period of interest (e.g., six months following diagnosis or treatment, respectively), and then dividing the population into two groups: a first group of subjects that progress to an endpoint (e.g., recurrence of disease, death); and a second group of subjects that did not progress to the end point. These are used to establish “low risk” and “high risk” population values for the marker(s) measured, respectively. Other suitable endpoints include, but are not limited to, 5-year mortality rates or progression to metastatic disease.

Once these groups are established, one or more thresholds may be selected that provide an acceptable ability to predict diagnosis, prognostic risk, treatment success, etc. In practice, Receiver Operating Characteristic curves, or “ROC” curves, are typically calculated by plotting the value of a variable versus its relative frequency in two populations (called arbitrarily “disease” and “normal” or “low risk” and “high risk” for example). For any particular marker, a distribution of marker levels for subjects with and without a disease may overlap. Under such conditions, a test does not absolutely distinguish “disease” and “normal” with 100% accuracy, and the area of overlap indicates where the test cannot distinguish “disease” and “normal.” A threshold is selected, above which (or below which, depending on how a marker changes with the disease) the test is considered to be “positive” and below which the test is considered to be “negative.” The area under the ROC curve is a measure of the probability that the perceived measurement may allow correct identification of a condition. See, e.g., Hanley et al., Radiology 143: 29-36 (1982).

Additionally, thresholds may be established by obtaining an earlier marker result from the same patient, to which later results may be compared. In some aspects, the individuals act as their own “control group.” In markers that increase with disease severity or prognostic risk, an increase over time in the same patient can indicate a worsening of disease or a failure of a treatment regimen, while a decrease over time can indicate remission of disease or success of a treatment regimen.

In certain embodiments, markers and/or marker panels can be selected to distinguish “disease” and “normal” or, alternatively “low risk” from “high risk” with at least about 70% sensitivity, at least about 80% sensitivity, at least about 85% sensitivity, at least about 90% sensitivity, or at least about 95% sensitivity, and combined with at least about 70% specificity, at least about 80% specificity, at least about 85% specificity, at least about 90% specificity, or at least about 95% specificity. In some embodiments, both the sensitivity and specificity can be at least about 75%, at least about 80%, at least about 85%, at least about 90%, or at least about 95%. The term “about” in this context refers to +/−5% of a given measurement.

In other embodiments, a positive likelihood ratio, negative likelihood ratio, odds ratio, and/or hazard ratio is used as a measure of a test's ability to predict disease, prognostic risk, or treatment outcome. In the case of a positive likelihood ratio, a value of 1 indicates that a positive result is equally likely among subjects in both a first group and a second group; a value greater than 1 indicates that a positive result is more likely in the first group; and a value less than 1 indicates that a positive result is more likely in the second group. In the case of a negative likelihood ratio, a value of 1 indicates that a negative result is equally likely among subjects in both groups; a value greater than 1 indicates that a negative result is more likely in the first group; and a value less than 1 indicates that a negative result is more likely in the second group. In certain embodiments, markers and/or marker panels may be selected to exhibit a positive or negative likelihood ratio of at least about 1.5 or more or about 0.67 or less, or at least about 2 or more or about 0.5 or less, or at least about 5 or more or about 0.2 or less, or at least about 10 or more or about 0.1 or less, or at least about 20 or more or about 0.05 or less. The term “about” in this context refers to +/−5% of a given measurement.

In the case of an odds ratio, a value of 1 indicates that a positive result is equally likely among subjects in both the first and second groups; a value greater than 1 indicates that a positive result is more likely in the first group; and a value less than 1 indicates that a positive result is more likely in the second group. In certain embodiments, markers and/or marker panels may be selected to exhibit an odds ratio of at least about 2 or more or about 0.5 or less, or at least about 3 or more or about 0.33 or less, or at least about 4 or more or about 0.25 or less, or at least about 5 or more or about 0.2 or less, or at least about 10 or more or about 0.1 or less. The term “about” in this context refers to +/−5% of a given measurement.

In the case of a hazard ratio, a value of 1 indicates that the relative risk is equal in both the first and second groups; a value greater than 1 indicates that the risk is greater in the first group; and a value less than 1 indicates that the risk is greater in the second group. In certain embodiments, markers and/or marker panels may be selected to exhibit a hazard ratio of at least about 1.1 or more or about 0.91 or less, or at least about 1.25 or more or about 0.8 or less, or at least about 1.5 or more or about 0.67 or less, or at least about 2 or more or about 0.5 or less, or at least about 2.5 or more or about 0.4 or less. The term “about” in this context refers to +/−5% of a given measurement.

In some embodiments, multiple thresholds may be determined. This can be the case in so-called “tertile,” “quartile,” or “quintile” analyses. In these methods, the “disease” and “normal” groups (or “low risk” and “high risk”) groups can be considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In some embodiments, assays can be “configured to detect” a particular marker. In one embodiment, an assay can generate a detectable signal indicative of the presence or amount of a physiologically relevant concentration of that marker. As discussed in detail herein, an assay that is “configured to detect” a marker may also detect other “related” markers. Assays can be immunoassays, and an assay “configured to detect” NHERF-1 can detect at least intact NHERF-1, and may also detect one or more immunologically detectable fragments of NHERF-1. In other embodiments, assays may be configured to detect one or more markers related to NHERF-1, but not full length NHERF-1 itself. The terms “related markers” and “markers related thereto” are defined hereinafter.

In some embodiments, devices configured to perform one or more of the methods described herein are provided. Such devices may comprise at least one diagnostic zone configured to bind for detecting NHERF-1 and/or one or more markers related thereto. Such devices may comprise additional diagnostic zones configured to bind for the detection of other markers, and such diagnostic zones may be discrete locations within a single assay device. Such devices are often referred to as “arrays” or “microarrays.” Following reaction of a sample with the devices, a signal is generated from the diagnostic zone(s), which may then be correlated to the presence or amount of the markers of interest. Numerous suitable devices are known to those of skill in the art.

In certain embodiments, NHERF-1 may be combined with additional markers. The methods may comprise comparing each marker to a respective threshold value, or multiple assay results may be combined into a single composite value. In such methods, the composite result may be compared to a threshold value, rather than the individual assay results. Markers that may find use in combination with NHERF-1 in the methods described herein include, but are not limited to, podocalyxin-like protein 1, carcinoembryonic antigen (CEA), tissue polypeptide antigen (TPA), squamous carcinoma antigen (SCC-ag), ferritin, soluble interleukin-2 receptor (sIL-2r), chromagranin A, neuron-specific enolase (NSE), creatine kinase-BB (CK-BB), glycosyl transferase, bombesin/gastrin releasing peptide, adrenocorticotropin (ACTH), antidiuretic hormone (ADH), calcitonin, insulin-like growth factor-I (IGF-I), osteopontin, human epididymis protein 4 (HE4), and insulin-like growth factor-II (IGF-II). In addition, NHERF-1, alone or with these other markers, may also be combined with the results of additional medical studies such as X-ray, computed tomography (CT), magnetic resonance imaging (MRI), and biopsy to arrive at a diagnosis, prognosis, or treatment result.

### BRIEF DESCRIPTION OF THE DRAWINGS

The novel features of various aspects and embodiments of the invention are set forth with particularity in the appended claims. A better understanding of the features and advantages of the present invention may be obtained by reference to the following detailed description that sets forth illustrative aspects, in which principles of various aspects and embodiments of the invention are utilized, and the accompanying drawings of which:

FIG. 1 depicts a Receiver Operator Characteristic curve for the identification of ovarian cancer using NHERF-1;

FIG. 2 depicts a Receiver Operator Characteristic curve (for female patients only) for the identification of ovarian cancer using NHERF-1;

FIG. 3 depicts a Receiver Operator Characteristic curve (for all patients, including male normal donors) for the identification of ovarian cancer using NHERF-1;

FIG. 4 depicts a Receiver Operator Characteristic curve for the identification of lung cancer using NHERF-1; and

FIG. 5 depicts a Receiver Operator Characteristic curve for the identification of lung cancer using NHERF-1.

### DETAILED DESCRIPTION OF THE INVENTION

While certain aspects and embodiments of the invention have been shown and described herein, it will be obvious to those skilled in the art that such aspects and embodiments are provided by way of example only. Numerous variations, changes, and substitutions will now occur to those skilled in the art without departing from the invention. It should be understood that various alternatives to aspects and embodiments of the invention described herein may be employed. It is intended that the claims define the scope of the invention and that methods and structures within the scope of these claims and their equivalents be covered thereby.

### DEFINITIONS

The term “NHERF-1” as used herein refers to a mature polypeptide described in Swiss-Prot accession number O14745 or its non-human homologue. Human NHERF-1 has the following sequence:

```
(SEQ ID NO: 1)
                10 20 30 40
MSADAAAGAP LPRLCCLEKG PNGYGFHLHG EKGKLGQYIR
                50 60 70 80
LVEPGSPAEK AGLLAGDRLV EVNGENVEKE THQQWSRIR
                90 100 110 120
AALNAVRLLV VDPETDEQLQ KLGVQVREEL LRAQEAPGQA
              130 140 150 160
EPPAAAEVQG AGNENEPREA DKSHPEQREL RPRLCTMKKG
              170 180 190 200
PSGYGFNLHS DKSKPGQFIR SVDPDSPAEA SGLRAQDRIV
              210 220 230 240
EVNGVCMEGK QHGDWSAIR AGGDETKLLV VDRETDEFFK
              250 260 270 280
KCRVIPSQEH LNGPLPVPFT NGEIQKENSR EALAEAALES
              290 300 310 320
PRPALVRSAS SDTSEELNSQ DSPPKQDSTA PSSTSSSDPI
              330 340 350
LDFNISLAMA KERAHQKRSS KRAPQMDWSK KNELFSNL.
```

As noted in the Swiss-Prot annotation, mature NHERF-1 is believed to lack the initiation methioninc (met₁ in SEQ ID NO:1), and may be post-translationally modified (by N-acetylation of ser₂ and phosphorylation of ser₂₈₀, ser₂₉₀, ser₂₉₁, thr₂₉₃, and ser₂₉₄). Thus, NHERF-1 includes both the unmodified polypeptide and forms having one or more of these post-translational modifications.

The term “marker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments.

The terms “related marker” and “marker related thereto” as used herein refers to one or more immunologically detectable fragments of a particular marker or its biosynthetic parent that comprise 8 or more contiguous residues of the marker or its parent.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay.

The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. The presence, absence, amount, or change in amount of one or more markers may indicate that a particular disease is present, or may indicate that a particular disease is absent. NHERF-1 is a subject-derived marker.

markers can also include clinical “scores” such as a pre-test probability assignment, a pulmonary hypertension “Daniel” score, an NIH stroke score, a Sepsis Score of Elebute and Stoner, a Duke Criteria for Infective Endocarditis, a Mannheim Peritonitis Index, an “Apache” score, etc.

The term “test sample” as used herein refers to a sample of bodily fluid obtained for the purpose of diagnosis, prognosis, or evaluation of a subject of interest, such as a patient. In certain aspects, such a sample may be obtained for the purpose of determining the outcome of an ongoing condition or the effect of a treatment regimen on a condition. Test samples can include blood, serum, plasma, cerebrospinal fluid, urine, saliva, sputum, and pleural effusions. One of skill in the art would realize that some test samples would be more readily analyzed following a fractionation or purification procedure, for example, separation of whole blood into serum or plasma components. In various embodiments, a test sample can be blood or one of its fluid components (plasma or serum).

Assays may be “configured to detect” a particular marker, for example, NHERF-1. Because an antibody epitope is on the order of 8 amino acids, an immunoassay may detect other polypeptides (e.g., related markers) so long as the other polypeptides contain the epitope(s) necessary to bind to the antibody used in the assay. Such other polypeptides are referred to as being “immunologically detectable” in the assay.

That an assay is “configured to detect” a marker means that an assay can generate a detectable signal indicative of the presence or amount of a physiologically relevant concentration of a particular marker of interest. Such an assay may, but need not, specifically detect a particular marker (i.e., detect a marker but not some or all related markers). For example, forms of NHERF-1 cleaved by circulating proteases may comprise a large number of residues in common with NHERF-1, so an assay that is configured to detect NHERF-1 could also detect one or more of these NHERF-1-related forms. In the alternative, assays may be developed that are specific for one or more forms, in that other forms are not appreciably detected in the assay.

The term “diagnosis” refers to a relative probability that a certain disease is present in the subject, and not the ability of a “specific marker” to give a definitive yes/no answer to the existence of a disease. Similarly, the term “prognosis” refers to a relative probability that a certain future outcome may occur in the subject, and not the ability of a “specific marker” to give a definitive yes/no answer to the future outcome.

Accordingly, the terms “correlating” and “relating” as used herein in reference to the use of markers refers to comparing the presence or amount of the marker(s) in a patient to its presence or amount in persons known to suffer from, or known to be at risk of, a given condition, or in persons known to be free of a given condition, and assigning an increased or decreased probability of a particular diagnosis, prognosis, etc., to an individual based on the assay result(s) obtained from that individual. Relating an assay result to the presence or absence of ovarian cancer or lung cancer is not meant to indicate that the assay result(s) may have a level of sensitivity and specificity that meets the ideal of 100%. Moreover, the artisan understands that markers need not be elevated in a single specific condition for such markers to be useful to the artisan in clinical diagnosis. Few, if any, such definitive tests exist.

When a NHERF-1 assay is used in isolation, relating the assay results to a diagnosis or prognosis may mean comparing the measured assay result to a predetermined NHERF-1 threshold arrived at by examining a population of “normal” and “diseased” subjects and selecting a threshold that provides an acceptable level of sensitivity and specificity, an acceptable odds ratio, etc. A greater probability of particular diagnosis, prognosis, etc., is assigned to the subject above the threshold, relative to that which would be assigned below the threshold. That probability may be measured qualitatively (e.g., the subject is at an increased risk of having ovarian cancer above the threshold than below the threshold”) or quantitatively (e.g., “the odds ratio for the subject having lung or ovarian cancer is 5-fold higher above the threshold than below the threshold”). Alternatively, a “quartile” approach may be used, where the probability of particular diagnosis, prognosis, etc. is assigned based on into which bin of the quartile the measured assay result falls. Numerous other ways to express the relationship of the assay results to a diagnosis or prognosis are known in the art.

A marker level in a subject's sample can be compared to a level known to be associated with a diagnosis of cancer (e.g., ovarian cancer, lung cancer). The sample's marker level is the to have been correlated with a diagnosis; that is, the skilled artisan can use the marker level to determine whether the patient likely suffers from a specific type diagnosis, and respond accordingly. Alternatively, the sample's marker level can be compared to a marker level known to be associated with a good outcome (e.g., the absence of ovarian cancer, etc.) in a “rule out” approach. In various embodiments, a profile of marker levels can be correlated to a global probability or a particular outcome using ROC curves.

As used herein, a “plurality” as used herein refers to at least 2, or at least 3, or at least 5, or at least 10, or at least 15, or at least 20. In some embodiments, a plurality can be a large number, i.e., at least 100.

The term “discrete” as used herein refers to areas of a surface that are noncontiguous. That is, two areas are discrete from one another if a border that is not part of either area completely surrounds each of the two areas.

The term “subject” as used herein refers to a human or non-human organism. Thus, the methods and compositions described herein are applicable to both human and veterinary disease. Further, while a subject can be a living organism, in some embodiments the term may refer to post-mortem analysis. In some embodiments, a subject may be a “patient,” i.e., living humans that are receiving medical care for a disease or condition. This includes persons with no defined illness who are being investigated for signs of pathology, and persons being evaluated for the presence of cancer (e.g., ovarian cancer, lung cancer).

The term “independently addressable” as used herein refers to discrete areas of a surface from which a specific signal may be obtained.

The term “therapy regimen” refers to one or more interventions made by a caregiver in hopes of treating a disease or condition.

Various aspects of the invention relate to methods and compositions for symptom-based differential diagnosis of lung or ovarian cancer, prognosis of lung or ovarian cancer, and monitoring of treatment regimens in subjects having lung or ovarian cancer. In particular, certain aspects relate to methods and compositions using the protein NHERF-1 as a diagnostic and prognostic marker in lung or ovarian cancer. As will be clear to the skilled artisan, the methods and devices described herein may be employed with respect to ovarian cancer, lung cancer, or both lung and ovarian cancer.

Aspects and embodiments of the invention can be used with all types of lung or ovarian cancer. The types of ovarian cancer in which aspects and embodiments of the invention can be applied include, without limitation, serous and non-serous, such as endometrioid, mucinous and clear cell. The types of lung cancer in which aspects and embodiments of the invention can be applied include, without limitation, primary lung cancer, such as small cell lung cancer and non-small cell lung cancer (e.g., squamous cell carcinoma, adenocarcinoma and large cell carcinoma), mesothelioma and secondary lung cancer.

Patients presenting for medical treatment for ovarian cancer often exhibit one or a few primary observable changes in bodily characteristics or functions that are indicative of disease. Often, these “symptoms” are nonspecific, in that a number of potential diseases can present the same observable symptom or symptoms. In the case of ovarian cancer, many women, including those with advanced cancer, experience only rather bland symptoms such as dyspepsia, bloating, early satiety, gas pains, and backache, and early cancer is usually asymptomatic. Pelvic pain, anemia, cachexia, and abdominal swelling due to ovarian enlargement or ascites usually occur later in advanced disease. Ovarian cancer may be suspected in women with unexplained adnexal masses, unexplained abdominal bloating, changes in bowel habits, unintended weight loss, or abdominal pain.

Aspects of the present invention describe methods and compositions that can assist in the differential diagnosis of such nonspecific symptoms by providing diagnostic markers that are designed to rule in or rule out one or a plurality of possible etiologies for the observed symptoms. Symptom-based differential diagnosis described herein can be achieved using panels of diagnostic markers designed to distinguish between possible diseases that underlie a nonspecific symptom observed in a patient.

### Selecting a Threshold

The skilled artisan understands that even for biomarkers that are routinely used in the medical setting, the performance characteristics, such as the desired specificity and sensitivity, appropriate thresholds, etc., for the particular test and patient population under study, must be established by the skilled artisan. Two or more assays for a particular biomarker may not yield the same results for immunoassays. That is, a threshold concentration selected for a particular assay platform may not translate to a different assay platform. For example, in the case of cardiac troponin I (a marker of myocardial damage commonly assayed in clinical laboratories), it has been reported that measurements using different commercial FDA-approved troponin I assays on identical specimens may differ in measured concentration by 100-fold. See, e.g., Christenson et al., “Standardization of Cardiac Troponin I Assays: Round Robin of Ten Candidate Reference Materials,” Clin. Chem. 47: 431-37 (2001). Thus, in developing a particular marker test, the skilled artisan understands that appropriate thresholds need to be determined for that particular test, and certain well-established methods can be used to do so.

In one embodiment, levels of the marker(s) being employed are obtained from a group of subjects that is divided into at least two sets. The first set includes subjects who have been confirmed as having a disease, outcome, or, more generally, being in a first condition state. For example, this first set of patients may be those diagnosed with cancer (diagnosis group), those that suffer a recurrence of cancer (prognosis group), or those that enter remission following treatment for cancer (therapy group). Subjects in this first set can be referred to as “diseased.” The second set of subjects is simply those who do not fall in the first set. Subjects in this second set can be referred to as “non-diseased.” The second set may be normal patients, and/or patients that do not suffer from recurrence, and/or that are refractory to treatment. In embodiments, the first set and the second set each have an approximately equal number of subjects.

In addition, serial testing of a marker in the same patient may also be used to establish a threshold. In effect, an earlier assay result from the same patient acts as a threshold to which later results may be compared. For example, serial CA-125 levels may identify cases better than a fixed CA-125 cutoff for identifying the likelihood of ovarian cancer. See, e.g., Skates et al., J. Clin. Oncol. 21(10 Suppl):206-10, 2003. It has been reported that recurrence of clinical stage I nonsmall-cell lung cancer can be predicted by decreasing levels of E-selectin, increasing levels of CD44, and increasing levels of urokinase plasminogen activator receptor. D'Amico et al., Ann. Thoracic Surg. 81: 1982-87, 2006. Similarly, many studies have shown that CA-125 levels frequently rise prior to clinical evidence of progression of ovarian cancer, and so serial measurements in the same patient can be used to determine prognosis and monitor the effectiveness of treatment. See, e.g., Rustin et al., Ann. Oncol. 7:361-364, 1996.

As noted above, a single marker often is incapable of definitively identifying a subject as falling within a first or second group. For example, if a patient is measured as having a marker level that falls within an overlapping region in the distribution of diseased and non-diseased subjects, the results of the test may be useless in diagnosing the patient. A cutoff may be established to distinguish between a positive and a negative test result for the detection of the disease or condition. Regardless of where the cutoff is selected, the effectiveness of the single marker as a diagnosis tool is unaffected. Changing the cutoff can serve as a trade off between the number of false positives and the number of false negatives resulting from the use of the single marker.

The effectiveness of a test having such an overlap is often expressed using an ROC (Receiver Operating Characteristic) curve. ROC curves are well known to those skilled in the art. The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level may allow correct identification of a disease or condition. Thus, the area under the ROC curve can be used to determine the effectiveness of the test.

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, tests and assays can exhibit one or more of the following results on these various measures: at least 75% sensitivity, combined with at least 75% specificity;

ROC curve area of at least 0.6, at least 0.7, or at least 0.8, or at least 0.9, or at least 0.95; and/or
a positive likelihood ratio (calculated as sensitivity/(1-specificity)) of at least 5, at least 10, or at least 20, and a negative likelihood ratio (calculated as (1-sensitivity)/specificity) of less than or equal to 0.3, or less than or equal to 0.2, or less than or equal to 0.1.
Use of NHERF-1 in Combination with Other Clinical Indicia

Once obtained, the relationship of the assay results to a particular diagnosis or prognosis may be used in a variety of manners.

For example, a diagnosis indicating an increased risk of having ovarian cancer may require that the subject receive additional diagnostic tests. A variety of imaging techniques may be performed to determine the size, shape, location and consistency of the ovaries. These include, without limitation, ultrasound, or, more specifically, transvaginal ultrasonography or transvaginal sonography (TVS). TVS may not be capable of distinguishing cancerous ovarian masses from benign masses. However, the diagnostic potential of TVS can be improved when paired with marker tests, including NHERF-1 assays. Other imaging methods include computed tomography (CT) (contrast medium may be employed to highlight the intestines and emphasize any spread of cancer within the pelvic cavity), magnetic resonance imaging (MRI), and transvaginal color flow doppler (which measures blood flow to the ovaries). It will be appreciated that there are numerous methods by which marker results may be combined with such imaging studies. For example, an increased risk of a particular diagnosis or prognosis may be assigned to a subject based on an NHERF-1 concentration above some cutoff. That risk may be further increased if an imaging study also indicates an increased risk of the same diagnosis or prognosis, or may be decreased if an imaging study indicates a decreased risk of the same diagnosis or prognosis.

In addition to NHERF-1 and imaging studies, other tests may be used to help verify a diagnosis of ovarian cancer. These include analyses for other tumor markers, tests for genetic mutations, and the microscopic examination of ovarian cells. Thus, assays that detect one or more of the markers described below may be combined with the NHERF-1 assays described herein. For example, CA-125 may be combined with NHERF-1. As another example, α-Fetoprotein may be combined with NHERF-1.

CA-125 (or OC-125) is a blood protein known as a tumor marker. While roughly 85% of women with clinically apparent ovarian cancer have increased levels of CA-125 relative to the normal blood level, the level of CA-125 is also increased during the first trimester of pregnancy, during menstruation, and in the presence of noncancerous illnesses (e.g., liver failure, pelvic inflammatory disease, endometriosis) and cancers of other sites (e.g., breast, lung, pancreas, colorectal).

Carcinoembyonic antigen (CEA), α-Fetoprotein (AFP), and human chorionic gonadotropin (β-hCG) may be used in the diagnosis and with determining the success of treatments of germ cell ovarian cancers. Reportedly, median survival times are increased in patients having low concentrations of these markers relative to patients with one or more positive marker levels. See Koh and Cauchi, Aust. NZ J. Obstet. Gynaecol. 23: 69-72, 1983. Combined AFP and b-hCG testing is used in the evaluation and treatment of nonseminomatous germ cell tumors, and in monitoring the response to therapy.

Other markers that have been reported as diagnostic and/or prognostic markers in ovarian cancer include, without limitation, alpha-1-antitrypsin, alpha(v) integrin, alpha(v) beta(6) Integrin, ATP7B, beta-2-microglobulin), beta III tubulin, CA54/61, CA 72-4, CA125 II, caGT (cancer-associated galactosyltransferase antigen), CASA or YKL-40, cathepsin B, CD24, CD34, c-Etsl, creatine kinase B, COX-1, EMMPRIN (extracellular matrix metalloproteinase inducer), Ep-CAM (epithelial cell adhesion molecule), Ets-1, GAT (galactosyltransferase associated with tumor), GEP (granulin-epithelin precursor), GT-II (galactosyltransferase isozyme II), human epididymis protein 4 (HE4, WFDC2), HER-2, hK8 (human kallikrein 8), hK10 (human kallikrein 10), hK13 (human kallikrein 13), HLA-G, HNF-1β, IAP (immunosuppressive acidic protein), IGFBP-2, KLK9 (kallikrein gene 9), M-CAM (melanoma cell adhesion molecule), M-CSF (macrophage colony-stimulating factor), mesothelin, MMP-2 (matrix metalloproteinase-2), nm23-H1, osteopontin, p53, P-III-P (type III procollagen peptide), P-glycoprotein, PP-4 (mlacental protein 4), mrogesterone, progesterone receptor (PR), prostasin, PUMP-1, sialyl SSEA-1 antigen, SM047, STN antigen (serum sialyl Tn antigen), TAG-72, thymidine phosphorylase (TP), TNF Receptor p75, topoisomerase II, tPA (tissue plasminogen activator), VSGP/F-spondin, WT-1, YB-1 (Y box-binding protein-1), P-gp (P-glycoprotein), YKL-40 and podocalyxin-like protein 1.

Similarly, a diagnosis indicating an increased risk of having lung cancer may require that the subject receive additional diagnostic tests. A variety of imaging techniques may be performed to determine the size, shape, location and consistency of the lungs. These include, without limitation, computed conventional X-ray, tomography (CT), magnetic resonance imaging (MRI), and positron emission tomography (PET) imaging. The skilled artisan will understand that there are numerous methods by which marker results may be combined with such imaging studies. For example, an increased risk of a particular diagnosis or prognosis may be assigned to a subject based on an NHERF-1 concentration above some cutoff. That risk may be further increased if an imaging study also indicates an increased risk of the same diagnosis or prognosis, or may be decreased if an imaging study indicates a decreased risk of the same diagnosis or prognosis.

In addition to NHERF-1 and imaging studies, other tests may be used to help verify a diagnosis of lung cancer. These include analyses for other tumor markers, tests for genetic mutations, and the microscopic examination of lung cells. Thus, assays that detect one or more of the markers described below may be combined with the NHERF-1 assays described herein.

Numerous markers that have been reported as diagnostic and/or prognostic markers in lung cancer are summarized in, for example, Stieber et al., National Academy of Clinical Biochemistry Guidelines for the Use of Tumor Markers in Lung Cancer, in NACB: Practice Guidelines and Recommendations for use of tumor markers in the clinic. Section 3P, 2006; and Ferrigno et al., Eur. Respir. J. 7: 186-97, 1994. The following table, adapted from Stieber et al., summarizes some details about certain of these markers:

### NSE

Differential diagnosis of lung masses when biopsy is not available: in high levels high specificity for small cell carcinoma; in SCLC, additive information to ProGRP
Assessing prognosis. High levels predict adverse outcome in SCLC
Assessing prognosis. High levels predict adverse outcome in NSCLC
Monitoring therapy in SCLC
Monitoring therapy in advanced disease (NSCLC)
Detection of recurrent disease. Increasing kinetics indicate progressive disease in SCLC

### CEA

Differential diagnosis of lung masses when biopsy is not available; in high levels high specificity for adenocarcinoma; in NSCLC, additive information to CYFRA 21-1
Assessing prognosis. High levels predict adverse outcome in early and advanced stage NSCLC
Monitoring therapy in advanced disease (NSCLC and SCLC)
Detection of recurrent disease. Increasing kinetics indicate progressive disease in NSCLC, part, in adeno cancer.

### CYFRA 21-1

Differential diagnosis of lung masses when biopsy is not available: in high levels high specificity for squamous cell carcinoma; best marker for NSCLC
Assessing prognosis. High levels predict adverse outcome in early and advanced NSCLC
Assessing prognosis. High levels predict adverse outcome in SCLC
Monitoring therapy in advanced disease (NSCLC)
Early prediction of therapy response in advanced disease (NSCLC)
Detection of recurrent disease. Increasing kinetics indicate progressive disease in NSCLC, part in squamous cell cancer.

### ProGRP

Differential diagnosis of lung masses when biopsy is not available: in high levels high specificity for small cell carcinoma; best marker for SCLC; additive information to NSE
Assessing prognosis. High levels predict adverse outcome in SCLC
Monitoring therapy in SCLC
Detection of recurrent disease. Increasing kinetics indicate progressive disease in SCLC.

### SCCA

Differential diagnosis of lung masses when biopsy is not available: in high levels high specificity for squamous cell carcinoma; in SQC additive information to CYFRA 21-1
Abnormal levels are associated with a high probability of NSCLC, mainly squamous tumors Assessing prognosis. High levels predict adverse outcome in NSCLC

### CA125

Differential diagnosis of lung masses when biopsy is not available; in high levels relative specificity for adenocarcinoma, large cell carcinoma
Assessing prognosis in NSCLC. High levels predict adverse outcome in NSCLC
Monitoring therapy in advanced disease (NSCLC)
Early prediction of therapy response in advanced disease (NSCLC)

### Chromogranin A

Differential diagnosis of lung masses when biopsy is not available; particularly for neuroendocrine tumors
Assessing prognosis. High levels predict adverse outcome in SCLC and in neuroendocrine tumors
Monitoring therapy in neuroendocrine tumors

### HER2-neu

Not appropriate for differential diagnosis
Assessing prognosis. High levels predict adverse outcome in advanced NSCLC: conflicting data
Monitoring therapy in NSCLC not possible

### DNA Fragments

Assessing diagnosis; correlation with stage
Assessing prognosis. High levels predict adverse outcome
Monitoring therapy in advanced disease (NSCLC)
Early prediction of therapy response in advanced disease (NSCLC)
Detection of recurrent disease. Increasing kinetics indicate progressive disease in NSCLC

### TPA

Differential diagnosis of lung masses when biopsy is not available
Assessing prognosis. High preoperative levels predict adverse outcome in NSCLC

### TPS

Assessing diagnosis (inferior to CYFRA 21-1 and TPA); correlation with stage
Assessing prognosis. High levels predict adverse outcome in NSCLC
Assessing prognosis. High levels predict adverse outcome in SCLC
Monitoring therapy in advanced disease (NSCLC)
Early prediction of therapy response in SCLC
Detection of recurrent disease. Increasing kinetics indicate progressive disease in NSCLC.

### TU M2-PK

Assessing diagnosis; inconsistent data are available
Monitoring therapy in NSCLC and SCLC
Detection of recurrent disease. Increasing kinetics indicate progressive disease in NSCLC and SCLC.
   Abbreviations: CEA: carcinoembryonic antigen; CYFRA 21-1: cytokeratin 19 fragments; HER2-neu: shed form of Her2-receptor; NSE: neuron specific enolase; ProGRP: progastrin-releasing peptide; SCCA: squamous cancer cell antigen; TPA: tissue polypeptide antigen (fragments from cytokeratins 8, 18 and 19); TPS: tissue polypeptide specific-antigen (the specific M3 epitope of tissue polypeptide antigen); TU M2-PK: tumor M2 pyruvate kinase.

Other lung cancer markers include, without limitation, ferritin, soluble interleukin-2 receptor (sIL-2r), creatine kinase-BB (CK-BB), glycosyl transferase, bombesin/gastrin releasing peptide, adrenocorticotropin (ACTH), antidiuretic hormone (ADH), calcitonin, insulin-like growth factor-I (IGF-I), osteopontin, human epididymis protein 4 (HE4), insulin-like growth factor-II (IGF-II) and podocalyxin-like protein 1.

NHERF-1 Complex Markers

NHERF-1 may form a complex with one or more biological and/or organic species to form a NHERF-1-containing complex. In embodiments, assays that detect one or more markers of such a NHERF-1-containing complex may be used in the diagnosis of ovarian or lung cancer, prognosis of ovarian or lung cancer and monitoring of treatment regimens in subjects having ovarian or lung cancer. In embodiments, assay results can be related to the presence or absence of ovarian or lung cancer, to the likelihood of an outcome related to ovarian or lung cancer, and/or to the success or failure of treatment received by subjects having ovarian or lung cancer. In one embodiment, the assays can detect one or more NHERF-1 markers of the NHERF-1-containing complex. In another embodiment, the assays can detect one or more markers of a species complexed with NHERF-1. In yet another embodiment, the assays can detect one or more markers of NHERF-1 and one or more markers of species complexed with NHERF-1. In some embodiments, the assays employ antibodies to NHERF-1 as well as antibodies for a species complexed with NHERF-1. In other embodiments, the assays employ antibodies to a NHERF-1-containing complex.

In embodiments, the species that complex with NHERF-1 are selected from EZR (ezrin), RDX (radixin), MSN (moesin), PDGFRA (platelet-derived growth factor receptor, alpha polypeptide), PDGFRB (platelet-derived growth factor receptor, beta polypeptide), ADRB2 (adrenergic, beta 2), NOS2 (nitric oxide synthase 2), CFTR (cystic fibrosis transmembrane conductance regulator), ARHGAP17 (Rho GTPase activating protein 17), EPI64 (TBC1 domain family, member 10A), GNB2L1 (guanine nucleotide binding protein, beta polypeptide 2-like 1), OPRK1 (opioid receptor, kappa 1), GNAQ (guanine nucleotide binding protein, q polypeptide), CTNNB1 (catenin (cadherin-associated protein), beta 1), PLCB3 (phospholipase C, beta 3), PDZK1 (PDZ domain containing 1), PAG1 (phosphoprotein associated with glycosphingolipid microdomains 1), SLC4A7 (solute carrier family 4, sodium bicarbonate cotransporter, member 7), ATP6V1B1 (ATPase), HTR4 (5 hydroxytryptamine (serotonin) receptor 4), CLCN3 (Chloride channel protein 3), SLC9A3R2 (sodium-hydrogen exchanger regulatory factor 2) and podocalyxin-like protein 1.

In one embodiment, assays that detect one or more markers of a NHERF-podocalyxin-like protein 1 complex may be used in the diagnosis of ovarian cancer, prognosis of ovarian cancer and monitoring of treatment regimens in subjects having ovarian cancer. In another embodiment, assays that detect one or more markers of a NHERF-podocalyxin-like protein 1 complex may be used in the diagnosis of lung cancer, prognosis of lung cancer and monitoring of treatment regimens in subjects having lung cancer.

BRCA1 and BRCA2 are genes that may be mutated in subjects with breast or ovarian cancer. Women who are at high risk because of a positive family history of ovarian and/or breast cancer may be offered BRCA1 and BRCA2 mutation screening. Such screening can help establish the degree of risk in women for ovarian and/or breast cancer.

One skilled in the art will recognize that univariate analysis of markers can be performed and the data from the univariate analyses of multiple markers can be combined to form panels of markers to differentiate different disease conditions. Such methods include, without limitation, multiple linear regression, determining interaction terms and stepwise regression. In embodiments, marker panels combine results from multiple marker assays into a single composite result. This single composite result may be used as if it is a single marker, and so subjected to ROC analysis to select decision thresholds, etc. Suitable methods for identifying and using markers panels are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453.

Clinical data may also be combined using “classification trees” (also known as “decision trees”). Many statistical software packages (e.g., MATLAB, CART and SPSS) can be used for this purpose, given that the clinical data is in the format X(m,n) and R(n). The trees may be produced with a large variety of splitting rules, prior probabilities and weighting schemes. The trees maybe fit to an arbitrary level of detail, or pruned using various cross-validation methods to avoid over-fitting the data. Large ensembles of trees may also be combined, for example, via Bootstrap Aggregation. A multivariate logistic regression model may be fed as input (together with the biomarkers) to a decision tree algorithm, or vice versa, the node assignments of a decision tree model may be fed as input (together with the biomarkers) into multivariate logistic regression. Similarly, any of the models may be fed as one of the inputs (together with the biomarkers) to a Neural Network.

### Selecting and Monitoring a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. There are appropriate treatments for numerous diseases discussed in relation to the methods of diagnosis described herein. See, e.g., Merck Manual of Diagnosis and Therapy, 17th Ed. Merck Research Laboratories, Whitehouse Station, N.J., 1999. With regard to SIRS, sepsis, severe sepsis, and septic shock, recent guidelines provide additional information for the clinician. See, e.g., Dellinger et al., Crit. Care Med. 32: 858-73, 2004.

Treatment for ovarian cancer includes, without limitation, surgery to remove cancerous tissue, chemotherapy and radiotherapy. In the United States, the initial treatment of ovarian cancer is now in transition, with most patients receiving primary therapy with drugs that contain platinum and taxane compounds (e.g., cisplatin, carboplatin, paclitaxel). However, other drugs, such as melphalan and anthracyclines, may also be used. The dose, timing and choice of chemotherapies can be determined by factors such as the type and stage of ovarian cancer, response to and recovery from chemotherapy, and health status.

Subject-derived markers of ovarian cancer may be analyzed in order to monitor the effectiveness of therapy. For example, remission is most likely among patients whose CA-125 levels drop below a normal value before their third chemotherapy treatment.

Treatment for lung cancer includes, without limitation, surgery to remove cancerous tissue; chemotherapy; and radiotherapy. As described above, subject derived markers of lung cancer are often analyzed in order to monitor the effectiveness of therapy.

### Assay Measurement Strategies

Numerous methods and devices are available for the detection and analysis of markers described in various aspects and embodiments of the invention. With regard to polypeptides or proteins in patient test samples, immunoassay devices and methods can be used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377. For separate or sequential assay of markers, suitable apparatuses include clinical laboratory analyzers such as the ELECSYS® (Roche), the AXSYM® (Abbott), the ACCESS® (Beckman), the AD VIA® CENTAUR® (Bayer) immunoassay systems, the NICHOLS ADVANTAGE® (Nichols Institute) immunoassay system, etc.

In certain embodiments, markers can be analyzed using an immunoassay or a sandwich immunoassay, although other methods are known to those skilled in the art. The presence or amount of a marker is generally determined using antibodies specific for each marker and detecting specific binding. Any suitable immunoassay, such as, for example, enzyme-linked immunoassays (ELISA), radioimmunoassays (RIAs), and competitive binding assays, may be utilized. Specific immunological binding of the antibody to the marker can be detected directly or indirectly. Direct labels include fluorescent or luminescent tags, metals, dyes, radionuclides, and the like, attached to an antibody. Indirect labels include various enzymes known in the art, such as, e.g., alkaline phosphatase and horseradish peroxidase.

In embodiments, apparatuses perform simultaneous assays of a plurality of markers using a single test device. Particularly useful physical formats comprise surfaces having a plurality of discrete, addressable locations for the detection of a plurality of different analytes. Such formats include protein microarrays, or “protein chips” (see, e.g., Ng and Hag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analytes (e.g., a marker) for detection at each location. Surfaces may alternatively comprise one or more discrete particles (e.g., microparticles, nanoparticles) immobilized at discrete surface locations. The particles can comprise antibodies configured to immobilize an analyte (e.g., a marker) for detection.

In some embodiments, immobilized marker-specific antibodies can be used. The marker-specific antibodies could be immobilized onto a variety of solid supports, such as, e.g., magnetic or chromatographic matrix particles, the surface of an assay place (such as microtiter wells) and pieces of a solid substrate material or membrane (such as, e.g., plastic, nylon, paper). An assay strip could be prepared by coating the marker-specific antibody or a plurality of marker-specific antibodies in an array on a solid support. This strip could then be dipped into the test sample and then processed through washes and detection steps to generate a measurable signal, such as a colored spot.

In some embodiments, devices of the present invention can comprise, for one or more assays, a first antibody conjugated to a solid phase and a second antibody conjugated to a signal development element. Such assay devices can be configured to perform a sandwich immunoassay for one or more analytes. These assay devices can further comprise a sample application zone and a flow path from the sample application zone to a second device region comprising the first antibody conjugated to a solid phase.

Flow of a sample along the flow path may be driven passively (e.g., by capillary, hydrostatic, or other forces that do not require further manipulation of the device once a sample is applied), actively (e.g., by application of force generated via mechanical pumps, electroosmotic pumps, hydrostatic pumps, centrifugal force, increased air pressure), or by a combination of active and passive driving forces. In some embodiments, a sample applied to the sample application zone can contact both a first antibody conjugated to a solid phase and a second antibody conjugated to a signal development element along the flow path (sandwich assay format). It will be appreciated that additional elements, such as, e.g., filters to separate plasma or serum from blood and mixing chambers, can be included. Exemplary devices are described in Chapter 41, entitled “Near Patient Tests: TRIAGE® Cardiac System,” in The Immunoassay Handbook, 2nd ed., David Wild, ed., Nature Publishing Group, 2001.

The analysis of markers could be carried out in a variety of physical formats as well. For example, the use of microtiter plates or automation could be used to facilitate the processing of large numbers of test samples. Alternatively, in certain embodiments (e.g., in ambulatory transport, emergency room settings), single sample formats could be developed to facilitate immediate treatment and diagnosis in a timely fashion.

A panel comprising one or more of the markers described above may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out to optimize clinical sensitivity or specificity in various clinical settings. These include, but are not limited to, ambulatory, urgent care, critical care, intensive care, monitoring unit, inpatient, outpatient, physician office, medical clinic and health screening settings. Furthermore, a single marker or a subset of markers comprising a larger panel of markers in combination with an adjustment of the diagnostic threshold can be used in each of the aforementioned settings to optimize clinical sensitivity and specificity. The clinical sensitivity of an assay can be defined as the percentage of those with the disease that the assay accurately predicts; the specificity of an assay can be defined as the percentage of those without the disease that the assay accurately predicts (see, e.g., Tietz Textbook of Clinical Chemistry, 2nd edition, Carl Burtis and Edward Ashwood eds., W.B. Saunders and Company, p. 496).

In certain embodiments, a kit for the analysis of markers is provided. can. Such a kit can comprise devices and reagents for the analysis of at least one test sample and instructions for performing the assay. Optionally, the kit may contain one or more means for using information obtained from immunoassays performed for a marker panel to rule in or rule out certain diagnoses. Other measurement strategies applicable to the methods described herein include, without limitation, chromatography (e.g., HPLC), mass spectrometry, x-ray photoelectron spectroscopy (XPS), receptor-based assays, and combinations of the foregoing.

### Selection of Antibodies

The generation and selection of antibodies may be accomplished in several ways. For example, one way is to purify polypeptides of interest or to synthesize the polypeptides using, e.g., solid phase peptide synthesis methods available in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; and Fujiwara et al., Chem. Pharm. Bull. (Tokyo) 44: 1326-31, 1996. The selected polypeptides may then be injected into various subjects (e.g., mice, rabbits) to generate polyclonal or monoclonal antibodies. One skilled in the art will recognize that many procedures are available for the production of antibodies. See, e.g., Antibodies, A Laboratory Manual, Ed Harlow and David Lane, Cold Spring Harbor Laboratory (1988), Cold Spring Harbor, N.Y. One skilled in the art will appreciate that binding fragments or Fab fragments which mimic antibodies can also be prepared from genetic information by various procedures. See, e.g., Antibody Engineering: A Practical Approach (Borrebaeck, C, ed.), 1995, Oxford University Press, Oxford; J. Immunol. 149, 3914-3920 (1992.

In addition, numerous publications have reported the use of phage display technology to produce and screen libraries of polypeptides for binding to a selected target. See, e.g., Cwirla et al., Proc. Natl. Acad. Sci. USA 87, 6378-82, 1990; Devlin et al., Science 249, 404-6, 1990; Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material can allow simultaneous mass screening of very large numbers of phage bearing different polypeptides. Phage displaying a polypeptide with affinity to a target can bind to the target, and these phage can be enriched by affinity screening to the target. The identity of polypeptides displayed from these phage can be determined from their respective genomes. Using these methods, a polypeptide identified as having a binding affinity for a desired target can then be synthesized in bulk by conventional means. See, e.g., U.S. Pat. No. 6,057,098.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies can then placed into the respective microtiter wells and incubated between about 30 minutes and 2 hours. The microtiter wells can then be washed and a labeled secondary antibody (for example, an anti-mouse antibody conjugated to alkaline phosphatase if the raised antibodies are mouse antibodies) can be added to the wells and incubated for about 30 minutes and then washed. A substrate can be added to the wells, and a color reaction may appear where one or more antibodies to the immobilized polypeptides are present.

The antibodies so identified may then be further analyzed for affinity and specificity in the assay design selected. In the development of immunoassays for a target protein, the purified target protein can act as a standard by which to judge the sensitivity and specificity of the immunoassay using the antibodies that have been selected. Because the binding affinity of various antibodies may differ; certain antibody pairs (e.g., in sandwich assays) may interfere with one another sterically. Assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides.

### EXAMPLES

The following examples serve to illustrate various aspects and embodiments of the invention. These examples are in no way intended to limit the scope of the invention.

### Example 1

### Cloning NHERF-1 into pET-41a(+) Bacterial Expression Vector

PCR primers A and B (5′ and 3′ respectively, Table 2) were made corresponding to the coding sequence at the 5′-end of the human NHERF-1 and the coding sequence at the 3′-end of human NHERF-1 (Genbank accession number NM_{—}004252.1). The 5′ primer also contained 21 nucleotides encoding a 7-histidine tag between the first amino acid and the second amino acid of NHERF-1. The histidine tag was used for purifying the recombinant protein. The 3′ primer contained an additional 22 base-pairs of pET-41a(+) vector sequence, including the Avr II site and sequence immediately downstream, at its 5′ end.

TABLE 1
PCR and Sequencing Primer Sequences:

| | |
|---|---|
| Primer A (SEQ ID NO: 2): | 5′ ATG CAT CAT CAC CAT |
| | CAC CAT CAC AGC GCG GAC |
| | GCA GCG GCC3′ |
| Primer B (SEQ ID NO: 3): | 5′ CGG GCT TTG TTT AGC |
| | AGC CTA G TTA TCA GAG |
| | GTT GCT GAA GAG TTC G3′ |

The PCR amplification of the NHERF-1 gene insert was done using Open Biosystems NHERF-1 cDNA (catalog #MHS1011-59107, Open Biosystems, Huntsville, Ala.) as template, PCR primers A and B, and AccuPrime Pfe DNA polymerase (Invitrogen, Carlsbad, Calif.) according to the manufacturer's recommendation. The reaction was carried out in an Applied Biosystems (Foster City, Calif.) thermal cycler using the cycling program recommended for the AccuPrime Pfx DNA polymerase.

An aliquot of the PCR product from the amplification using primers A and B was used as template for a second amplification using primers C (see below) and B. The 5′ primer C contained the coding sequence of the first amino acid of NHERF-1 and the sequence of the 7-histidine tag. Primer C also contained 21 base pairs of pET-41a(+) vector sequence (Novagen, Madison, Wis.) at its 5′-end corresponding to the NdeI site and sequence immediately upstream.

The vector sequence at the 5′-ends of these primers may form, upon treatment with T4 DNA polymerase, single-stranded overhangs that are specific and complementary to those on the vector.

The PCR products were prepared for agarose gel electrophoresis by purifying the DNA with PureLink PCR Purification Kit (Invitrogen, Carlsbad, Calif.) following the manufacturer's recommendation. The PCR products were then fractionated by agarose gel electrophoresis, and the full-length products were excised from the gel, purified, and resuspended in water using the QIAquick Gel Extraction Kit (Qiagen, Valencia, Calif.) following the manufacturer's recommendation.

The pET-41a(+) vector was prepared to receive insert by digestion with NdeI (New England BioLabs, Beverly, Mass.) and AvrII (New England BioLabs, Beverly, Mass.) according to manufacturer's recommendation. The NHERF-1 PCR insert and NdeI/AvrII digested pET-41a(+) vector were digested with T4 DNA polymerase (Roche Diagnostics, Indianapolis, Ind.) as described in Example 19 of U.S. Pat. No. 6,057,098. The T4 exonuclease digested insert and the digested pET-41a(+) vector were annealed, electroporated into electrocompetent E. coli strain, DH10B, and plated onto LB agar plates supplemented with kanamycin as described in Example 19 of U.S. Pat. No. 6,057,098. The sequence of the clones was verified using an Applied Biosystems 3130 Genetic Analyzer (Foster City, Calif.) according to the manufacturer's recommendation. DNA having the correct sequence was transformed into BL21(DE3) cells (Novagen, Madison, Wis.), and plated onto LB agar plates supplemented with kanamycin.

Primer C:
5′ CTT TAA GAA GGA GAT ATA CAT ATG CAT CAT CAC
CAT CAC CAT CAC 3′.

### Example 2

### Immunoassays

In general, for a sandwich immunoassay in microtiter plates, a monoclonal antibody directed against a selected analyte is biotinylated using N-hydroxysuccinimide biotin (NHS-biotin) at a ratio of about 5 NHS-biotin moieties per antibody. The antibody-biotin conjugate is then added to wells of a standard avidin 384 well microtiter plate, and antibody conjugate not bound to the plate is removed. This forms the “anti-marker” in the microtiter plate. Another monoclonal antibody directed against the same analyte is conjugated to alkaline phosphatase, for example using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Biotinylated antibodies are pipetted into microtiter plate wells previously coated with avidin and incubated for 60 min. The solution containing unbound antibody is removed, and the wells washed with a wash buffer, consisting of 20 mM borate (pH 7.42) containing 150 mM NaCl, 0.1% sodium azide, and 0.02% TWEEN®-20 surface active agent (ICI Americas). The plasma samples (e.g., 10 μL-20 μL) containing added HAMA inhibitors are pipetted into the microtiter plate wells, and incubated for 60 min. The sample is then removed and the wells washed with a wash buffer. The antibody-alkaline phosphatase conjugate is then added to the wells and incubated for an additional 60 min, after which time, the antibody conjugate is removed and the wells washed with a wash buffer. A substrate, (ATTOPHOS®, Promega, Madison, Wis.) is added to the wells, and the rate of formation of the fluorescent product is related to the concentration of the analyte in the sample tested.

For competitive immunoassays in microtiter plates, a murine monoclonal antibody directed against a selected analyte is added to the wells of a microtiter plate and immobilized by binding to goat anti-mouse antibody that is pre-absorbed to the surface of the microtiter plate wells (Pierce, Rockford, Ill.). Any unbound murine monoclonal antibody is removed after a 60 minute incubation. This forms the “anti-marker” in the microtiter plate. A purified polypeptide that is either the same as or related to the selected analyte, and that can be bound by the monoclonal antibody, is biotinylated as described above for the biotinylation of antibodies. This biotinylated polypeptide is mixed with the sample in the presence of HAMA inhibitors (human anti-mouse antibodies, or HAMA, are human immunoglobulins with specificity for mouse immunoglobulins; HAMA inhibitors may be used to reduce or eliminate false signals from these human immunoglobulins; see, e.g., Reinsberg, Clin. Biochem. 29:145-48, 1996), forming a mixture containing both exogenously added biotinylated polypeptide and any unlabeled analyte molecules endogenous to the sample. The amount of the monoclonal antibody and biotinylated marker added depends on various factors and is titrated empirically to obtain a satisfactory dose-response curve for the selected analyte.

This mixture is added to the microtiter plate and allowed to react with the murine monoclonal antibody for 120 minutes. After the 120 minute incubation, the unbound material is removed, and Neutralite-Alkaline Phosphatase (Southern Biotechnology; Birmingham, Ala.) is added to bind to any immobilized biotinylated polypeptide. Substrate (as described above) is added to the wells, and the rate of formation of the fluorescent product was related to the amount of biotinylated polypeptide bound, and therefore is inversely related to the endogenous amount of the analyte in the specimen.

### Example 3

### NHERF-1-Podocalyxin-Like Protein 1 Immunoassay

An indirect sandwich ELISA is used to detect a NHERF-1-podocalyxin-like protein 1 complex in patient samples. This may be used in the diagnosis of ovarian or lung cancer, prognosis of ovarian or lung cancer and monitoring of treatment regimens in subjects having ovarian cancer or lung.

In the indirect sandwich ELISA, biotinylated anti-NHERF-1 antibody (primary antibody), anti-NHERF-1-podocalyxin-like protein 1 complex antibody (primary antibody) and/or anti-podocalyxin-like protein 1 antibody (primary antibody) are diluted into assay buffer and allowed to incubate. Wells are washed with wash buffer, and samples and standards are subsequently added and allowed to incubate. Wells are washed again, and fluorsceinated anti-NHERF-1 antibody (secondary antibody), anti-NHERF-1-podocalyxin-like protein 1 complex antibody (secondary antibody) and/or anti-podocalyxin-like protein 1 antibody (secondary antibody) diluted in assay buffer are subsequently added and allowed to incubate at room temperature. Wells are washed again. Anti-fluorescein antibody conjugated to alkaline phosphatase, diluted into assay buffer, are added and allowed to incubate, followed by washing. Finally, substrate (Promega ATTOPHOS®) are added. Plates are read immediately

The plates are washed between each addition. Standards are prepared by spiking NHERF-1-podocalyxin-like protein 1 into a normal serum patient pool. Standards are run in 4 replicates, and samples are run singly. Reading is performed using a TECAN® Spectrafluor plus using kinetic mode reading of fluorescence. The assay slope (RFU/seconds) is determined and each sample concentration (NHERF-1-podocalyxin-like protein 1 complex concentration) is determined with reference to a 5 parameter log-logistic curve fit of the standard values.

### Example 4

### Study Population I

Samples were purchased through a commercial vendor and were collected from cancer patients from a site in Moscow, Russia. Samples were collected according to ProteoGenex Standard Collection Procedures, which comprise collecting blood samples using a Vacutainer SST tube (Becton Dickinson #366510 or VWR #VT6510). The tubes were inverted 5 times and allowed to clot at room temperature for 30 minutes (no more than 2 hours) then centrifuged for 10 minutes at 1300-1500×G at 4° C. Serum was then removed and transferred to polypropylene tubes and spun again. Serum was then transferred to cryovials and frozen and stored at −70° to −80° C. There were a total of 48 breast cancer patients, 72 colon cancer patients, 20 ovarian cancer patients and 19 prostate cancer patients.

TABLE 2
Ovarian cancer subject characteristics:

| | | TNM | |
|---|---|---|---|
| Patient ID | Histological Diagnosis | Classification | Grade |
| 03236 | serous adenocarcinoma | T3N0M1 | G3 |
| 03489 | serous adenocarcinoma | T3cNxM1 | G1 |
| 03502 | serous adenocarcinoma | TlcN0M0 | G2 |
| 03237 | serous adenocarcinoma | T3N0M0 | G1-3 |
| 03241 | adenocarcinoma | T3NcM0 | G3 |
| 03242 | adenocarcinoma | T3N0M0 | unknown |
| 03247 | adenocarcinoma | T3N0M0 | unknown |
| 03248 | serous adenocarcinoma | T2aN0M0 | G2 |
| 03249 | serous adenocarcinoma | T3bNlM0 | G2 |
| 03250 | endometrial cystadenocarcinoma | T2aN0M0 | G3 |
| 03251 | endometrial cystadenocarcinoma | TlcN0M0 | N/A |
| 03252 | serous adenocarcinoma | T3aN2M0 | G2 |
| 03253 | serous adenocarcinoma | T3bNlM0 | G2 |
| 03254 | serous adenocarcinoma | T3N1M0 | G3 |
| 03255 | endometrial cystadenocarcinoma | TlbN0M0 | G2 |
| 03257 | cystadenocarcinoma | T3cN0M0 | G3 |
| 03258 | cystadenocarcinoma | T2cN0M0 | G2 |
| 03259 | unknown | T3NxM0 | unknown |
| 03260 | endometrial cystadenocarcinoma | T2N0M0 | G2 |
| 03261 | serous adenocarcinoma | T2N0M0 | G2 |

Also included were 69 normal donors collected from the same institute. Information for each donor included: age, sex, race, tumor classification and grade of cancer, as well as smoking history, personal history and family history.

### Example 5

### NHERF-1 Immunoassay I

An indirect sandwich ELISA was used to detect NHERF-1 in patient samples. Antibodies for the ELISA were developed at Biosite using phage display methods. Biotinylated anti-NHERF-1 antibody (primary antibody) diluted into assay buffer (10 mM Tris, 150 mM NaCl, 1% BSA) to 2 μg/ml was added to a 384 Neutravidin coated plates (Pierce Product #NC19658) and allowed to incubate at room temperature for 1 hour. Wells were washed with wash buffer (20 mM Borate, 150 mM NaCl, 0.2% TWEEN®-20 surface active agent (ICI Americas)) and then samples and standards were added and allowed to incubate at room temperature for 1 hour. Wells again were washed and then fluorsceinated anti-NHERF-1 antibody (secondary antibody) diluted in assay buffer to 2 μg/ml was added and allowed to incubate at room temperature for 1 hour. Wells again were washed. Anti-fluorescein antibody conjugated to alkaline phosphatase, diluted 1/2338 into assay buffer was added and allowed to incubate at room temperature for 1 hour, followed by washing. Finally, substrate (Promega ATTOPHOS®) was added and plate was read immediately. All additions were 10 μL/well unless otherwise stated.

The plates were washed 3 times between each addition and final wash was 9 times. Standards were prepared by spiking NHERF-1 into a normal serum patient pool at concentrations ranging from 50 to 0.39 ng/ml, including a neutralized 0, which is the serum pool with excess concentration of each antibody used in the ELISA. Standards were run in 4 replicates, and samples were run singly. Reading was performed using a TECAN®Spectrafluor Plus using kinetic mode reading of fluorescence over 6 read cycles (excitation filter 430 nm and emission filter 570 nm). The assay slope (RFU/seconds) was determined and each sample concentration was determined by reference to a 5 parameter log-logistic curve fit of the standard values. Reported NHERF-1 concentrations are in ng/mL.

### Example 6

### Results I

The following summarizes the results of NHERF-1 measurements in ovarian cancer and normal subjects (n—number of subjects; mean—mean NHERF-1 concentration; median—median NHERF-1 concentration; SD—standard deviation; SE—standard error; 95%-95% confidence interval); IQR—interquartile range.

TABLE 3

| | | | SD | SE | 95% | | | 95% |
|---|---|---|---|---|---|---|---|---|
| Patient group | n | mean | (mean) | (mean) | (mean) | median | IQR | (median) |
| Ovarian cancer | 20 | 5.6 | 3.3 | 0.7 | 4.0-7.1 | 5.6 | 5.2 | 3.0-7.0 |
| Normal | 69 | 3.1 | 3.4 | 0.4 | 2.3-3.9 | 2.2 | 1.5 | 1.9-2.6 |

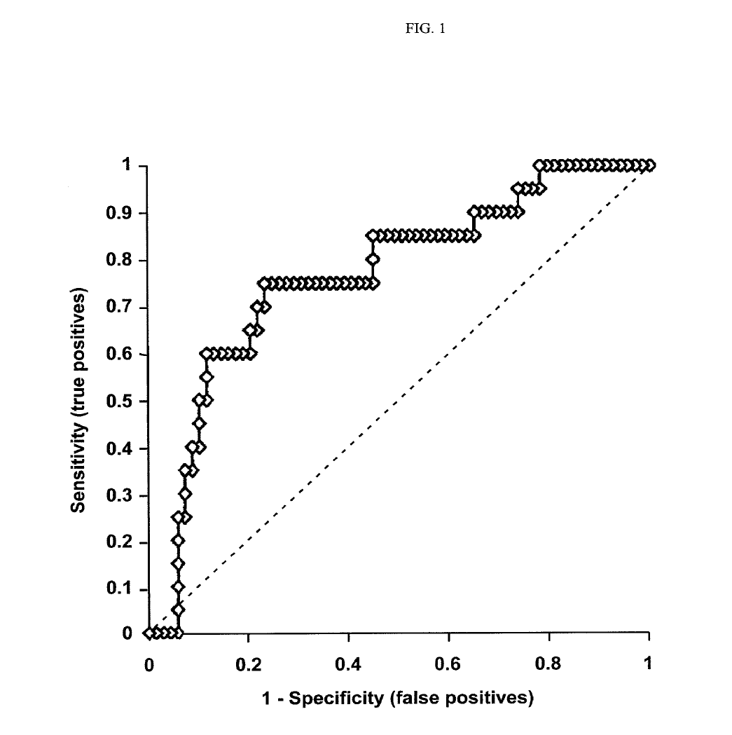

Using standard KOC analysis, the ability of NHERF-1 to distinguish ovarian cancer from normal was determined. The ROC area was 0.766 (95% confidence interval 0.65-0.89), giving a p value of <0.0001. This indicates that NHERF-1 is significantly increased in the ovarian cancer population. The ROC curve obtained is shown in FIG. 1. In addition, it was determined that NHERF-1 measurements in ovarian cancer were not correlated to CA 125 measurements.

Odds ratios may be calculated for the combination of ovarian cancer and normal data. In the following example, the odds ratio is defined as the ratio of the odds of an event occurring above a selected NHERF-1 concentration, relative to the odds of it occurring below that threshold. Three thresholds were selected: the 75th percentile concentration in normal subjects, the mean concentration of the combined normal and ovarian cancer population, and the median concentration in that population. Table 5 summarizes the results obtained.

TABLE 4

| | | Mean of | Median of |
|---|---|---|---|
| | 75th | Normal + | Normal + |
| | Percentile | Ovarian Cancer | Ovarian Cancer |
| Threshold Criteria: | of Normals | Cohorts | Cohorts |
| Threshold NHERF-1 | 2.9 | 3.7 | 2.5 |
| Concentration | | | |
| Odds Ratio | 8.5 | 7.1 | 3.9 |
| 95% confidence interval | 2.7-26.7 | 2.4-21.2 | 1.3-11.9 |
| p value, 2 tail (OR > 1): | 2.5 × 10⁻⁴ | 4.1 × 10⁻⁴ | 1.7 × 10⁻² |

These data indicate that the odds of having ovarian cancer are significantly increased in a subject if the NHERF-1 concentration measured in that subject exceeds any one of these threshold concentrations. Particularly striking is the fact that an individual having a concentration that exceeds the 75th percentile of normal has nearly a 9-fold greater probability of having ovarian cancer, compared to the probability when the concentration is less than the 75th percentile of normal.

### Example 7

### Distinguishing Ovarian Cancer from Breast and Colon Cancer

In addition to subjects having ovarian cancer and normal subjects, NHERF-1 concentrations were also measured in 48 breast cancer and 72 colon cancer subjects. The following summarizes the results obtained.

TABLE 5

| | | | SD | SE | 95% | | | 95% |
|---|---|---|---|---|---|---|---|---|
| Patient group | n | mean | (mean) | (mean) | (mean) | median | IQR | (median) |
| Ovarian | 20 | 5.6 | 3.3 | 0.7 | 4.0-7.1 | 5.6 | 5.2 | 3.0-7.0 |
| cancer | | | | | | | | |
| Breast cancer | 48 | 5.7 | 10.6 | 1.5 | 2.6-8.8 | 2.7 | 2.6 | 2.2-3.4 |
| Colon cancer | 72 | 3.3 | 3.5 | 0.4 | 2.5-4.2 | 2.4 | 1.9 | 1.9-2.9 |

Using standard ROC analysis, the ability of NHERF-1 to distinguish ovarian cancer from these other cancers was determined. The ROC area for distinguishing from breast cancer was 0.68 (95% confidence interval 0.54-0.82), giving a p value of <0.01. This indicates that NHERF-1 is significantly increased in the ovarian cancer population, relative to the breast cancer population. The ROC area for distinguishing from colon cancer was 0.75 (95% confidence interval 0.62-0.87), giving a p value of <0.0001. This indicates that NHERF-1 is also significantly increased in the ovarian cancer population, relative to the colon cancer population.

### Example 8

### Study Population II

Samples were purchased through a commercial vendor and were collected from cancer patients from a site in Moscow, Russia. Samples were collected according to ProteoGenex Standard Collection Procedures, which comprise collecting blood samples using a Vacutainer SST tube (Becton Dickinson #366510 or VWR #VT6510). The tubes were inverted 5 times and allowed to clot at room temperature for 30 minutes (no more than 2 hours) then centrifuged for 10 minutes at 1300-1500×G at 4° C. Serum was then removed and transferred to polypropylene tubes and spun again. Serum was then transferred to cryovials and frozen and stored at −70° to −80° C. There were a total of 71 breast cancer patients, 73 colon cancer patients, 33 ovarian cancer patients and 24 prostate cancer patients.

TABLE 6
Ovarian cancer subject characteristics:

| | | TNM | |
|---|---|---|---|
| Patient ID | Histological Diagnosis | Classification | Grade |
| 03235 | serous adenocarcinoma | T3N0M1 | G3 |
| 03236 | serous adenocarcinoma | T3N0M1 | G3 |
| 03237 | serous adenocarcinoma | T3N0M0 | G1-G3 |
| 03241 | serous adenocarcinoma | T3cN0M0 | G3 |
| 03242 | adenocarcinoma | T3N0M0 | unknown |
| 03243 | serious adenocarcinoma | T1cN0M0 | G2 |
| 03247 | adenocarcinoma | T3N0M0 | unknown |
| 03248 | serous adenocarcinoma | T2aN0M0 | G2 |
| 03249 | serous adenocarcinoma | T3bNlM0 | G2 |
| 03250 | endometrial cystadenocarcinoma | T2aN0M0 | G3 |
| 03251 | endometrial cystadenocarcinoma | TlcN0M0 | unknown |
| 03252 | serous adenocarcinoma | T3aN2M0 | G2 |
| 03253 | serous adenocarcinoma | T3bNlM0 | G2 |
| 03254 | serous adenocarcinoma | T3N1M0 | G3 |
| 03255 | endometrial cystadenocarcinoma | TlbN0M0 | G2 |
| 03257 | cystadenocarcinoma | T3cN0M0 | G3 |
| 03258 | cystadenocarcinoma | T2cN0M0 | G2 |
| 03259 | serous adenocarcinoma | T3N0M0 | unknown |
| 03260 | endometrial cystadenocarcinoma | T2N0M0 | G2 |
| 03261 | serous adenocarcinoma | T2N0M0 | G2 |
| 03267 | serous cystadenocarcinoma | T1bN0M0 | G2 |
| 03268 | serous cystadenocarcinoma | T1cN1M0 | G2 |
| 03269 | endometrial cystadenocarcinoma | T2N0M0 | G2 |
| 03270 | mucinous adenocarcinoma | T2N0M0 | unknown |
| 03271 | papillay cystadenocarcinoma | T3aN1M0 | unknown |
| 03274 | serous cystadenocarcinoma | T1bN0M0 | G2-G3 |
| 03275 | serous cystadenocarcinoma | T2N0M0 | G1 |
| 03276 | papillay cystadenocarcinoma | T1N0M0 | G2 |
| 03277 | serous cystadenocarcinoma | T2N0M0 | G2 |
| 03278 | serous cystadenocarcinoma | T1N0M0 | G2 |
| 03280 | serous cystadenocarcinoma | T2N0M0 | G1-G2 |
| 03489 | serous adenocarcinoma | T3cN0M1 | G1 |
| 03502 | serous adenocarcinoma | T1cN0M0 | G2 |

Also included were 37 normal female donors and 32 normal male donors collected from the same institute. Information for each donor included: age, sex, race, tumor classification and grade of cancer, as well as smoking history, personal history and family history.

### Example 9

### NHERF-1 Immunoassay II

An indirect sandwich assay using a Luminex assay platform was used to detect NHERF-1 in patient samples. Antibodies for the ELISA were developed at Biosite using phage display methods. Custom modified Luminex xMap™ magnetic beads covalently linked to an anti-NHERF-1 antibody (primary antibody) were diluted into assay buffer (about 50 mM Sodium Phosphate, 150 mM NaCL, 0.02% Tween20, 1% BSA) to about 50,000 beads/ml. Fifty μl of diluted beads were added to each well of a non-binding 96-well round bottom plate (Corning Product # 3605). Using a magnetic 96-well plate separator, the beads were pulled to the sides of the wells, washed and resuspended three times with 100 μl of assay buffer. The samples and standards were added to the beads and allowed to incubate for about 1 hour at room temperature on an orbital shaker. After the beads were washed and re-suspended again, biotinylated anti-NHERF-1 antibody (secondary antibody) diluted in assay buffer to about 0.05 μg/ml was added and allowed to incubate at room temperature for 1 hour on an orbital shaker. After washing and resuspension of the beads again, Streptavidin Phycoerthryin (PROzyme Phycolink Code #PJ31S) diluted to about 4 μg/ml in assay buffer was added and allowed to incubate for about 1 hour at room temperature on an orbital shaker. After the final wash and resuspension, the beads were passed through the flow cell of a Luminex 200 reader to measure assay signals.

The plates were washed 3 times between each addition and final wash was 9 times. Standards were prepared by spiking NHERF-1 into normal serum patient pool at concentrations ranging from about 100 ng/ml to 3.13 ng/ml, including a neutralized 0, which is the serum pool with excess concentrations of each antibody used in the sandwich assay. Standards were run in 2 replicates and samples were run singly. The assay median results taken from a minimum of 50-100 beads count signals was determined and each sample concentration was determined by reference to a 5 parameter log-logistic curve fit of the standard values. Reported NHERF-1 concentration are in ng/mL.

### Example 10

### Results II (for Female Patients Only)

The following summarizes the results of NHERF-1 measurements in ovarian cancer and normal subjects (limited to female patients only).

TABLE 7

| | | | SD | SE | 95% | | | 95% |
|---|---|---|---|---|---|---|---|---|
| Patient group | n | mean | (mean) | (mean) | (mean) | median | IQR | (median) |
| Ovarian cancer | 33 | 22.1 | 36.8 | 6.4 | 9.1-35.2 | 10.4 | 16.4 | 6.5-18.8 |
| Normal | 37 | 3.3 | 6.6 | 1.1 | 1.1-5.4 | 1.1 | 1.6 | 0.78-1.5 |

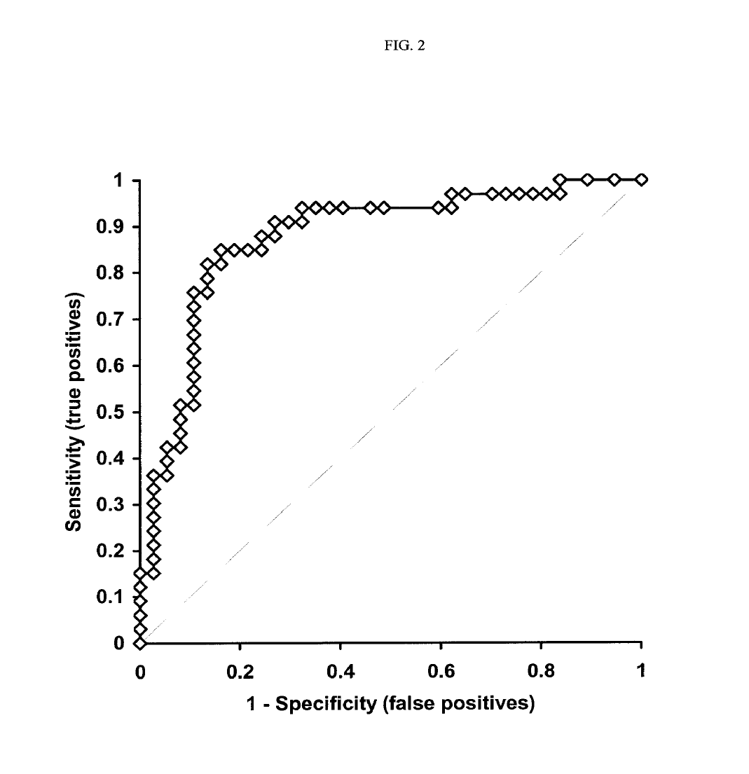

Using standard ROC analysis, the ability of NHERF-1 to distinguish ovarian cancer from normal was determined. The ROC area was 0.874 (95% confidence interval 0.788-0.961), giving a p value of <0.0001. This indicates that NHERF-1 is significantly increased in the ovarian cancer population. The ROC curve obtained is shown in FIG. 2. In addition, it was determined that NHERF-1 measurements in ovarian cancer were not correlated to CA 125 measurements.

The ROC area to distinguish serous ovarian cancer (n=21) from normal (n=35) was 0.880 (95% confidence interval 0.780-0.981), giving a p value of <0.0001. The ROC area to distinguish non-serous ovarian cancer (n=12) from normal (n=35) was 0.898 (95% confidence interval 0.786-1.008), giving a p value of <0.0001.

Odds ratios were calculated for the combination of ovarian cancer and normal data. Table 9 summarizes the results obtained (for female patients only).

TABLE 8

| | | Mean of | Median of |
|---|---|---|---|
| | 75th | Normal + | Normal + |
| | Percentile | Ovarian Cancer | Ovarian Cancer |
| Threshold Criteria: | of Normals | Cohorts | Cohorts |
| Threshold NHERF-1 | 2.3 | 12.1 | 2.7 |
| Concentration | | | |
| Odds Ratio | 22.6 | 12.9 | 28.9 |
| 95% confidence interval | 6.2-81.7 | 2.7-62.8 | 7.9-105.3 |
| p value, 2 tail (OR > 1): | 2.1 × 10⁻⁶ | 1.5 × 10⁻³ | 3.3 × 10⁻⁶ |

### Example 11

### Results II (for all Patients, Including Male Normal Donors)

The following summarizes the results of NHERF-1 measurements in ovarian cancer and normal subjects (for all patients, including male normal donors).

TABLE 9

| | | | SD | SE | 95% | | | 95% |
|---|---|---|---|---|---|---|---|---|
| Patient group | n | mean | (mean) | (mean) | (mean) | median | IQR | (median) |
| Ovarian cancer | 33 | 22.1 | 36.8 | 6.4 | 9.1-35.2 | 10.4 | 16.4 | 6.6-18.8 |
| Normal | 69 | 2.5 | 5.1 | 0.6 | 1.3-3.8 | 1.1 | 1.2 | 0.8-1.3 |

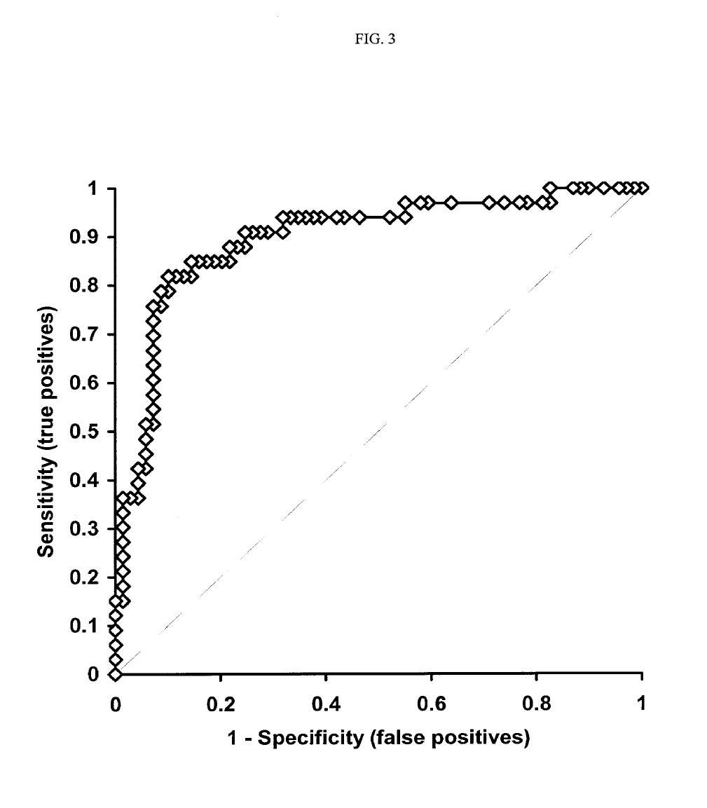

Using standard ROC analysis, the ability of NHERF-1 to distinguish ovarian cancer from normal was determined. The ROC area was 0.896 (95% confidence interval 0.826-0.965), giving a p value of <0.0001. This indicates that NHERF-1 is significantly increased in the ovarian cancer population. The ROC curve obtained is shown in FIG. 3. In addition, it was determined that NHERF-1 measurements in ovarian cancer were not correlated to CA 125 measurements.

Odds ratios were calculated for the combination of ovarian cancer and normal data. Table 11 summarizes the results obtained (for all patients, including male normal donors).

TABLE 10

| | | Mean of | Median of |
|---|---|---|---|
| | 75th | Normal + | Normal + |
| | Percentile | Ovarian Cancer | Ovarian Cancer |
| Threshold Criteria: | of Normals | Cohorts | Cohorts |
| Threshold NHERF-1 | 1.9 | 8.9 | 1.7 |
| Concentration | | | |
| Odds Ratio | 30.6 | 15.4 | 24.5 |
| 95% confidence interval | 8.3-113.0 | 4.9-49.0 | 6.7-89.5 |
| p value, 2 tail (OR > 1): | 2.9 × 10⁻⁷ | 2.6 × 10⁻⁶ | 1.0 × 10⁻⁶ |

### Example 12

### Distinguishing Ovarian Cancer from Breast and Colon Cancer (Female Subjects Only)

In addition to female subjects having ovarian cancer and normal female subjects, NHERF-1 concentrations were measured in 71 breast cancer and 35 colon cancer subjects (female subjects/patients only). The following summarizes the results obtained.

TABLE 11

| | | | SD | SE | 95% | | | 95% |
|---|---|---|---|---|---|---|---|---|
| Patient group | n | mean | (mean) | (mean) | (mean) | median | IQR | (median) |
| Ovarian | 33 | 22.1 | 36.8 | 6.4 | 9.1-35.2 | 10.4 | 16.4 | 6.5-18.8 |
| cancer | | | | | | | | |
| Breast cancer | 71 | 12.9 | 33.1 | 3.9 | 5.1-20.8 | 2.7 | 3.0 | 2.3-3.9 |
| Colon cancer | 35 | 3.9 | 5.1 | 0.9 | 2.1-5.6 | 1.6 | 4.5 | 0.9-3.2 |

Using standard ROC analysis, the ability of NHERF-1 to distinguish ovarian cancer from these other cancers was determined. The ROC area (for female patients only) for distinguishing from breast cancer was 0.75 (95% confidence interval 0.64-0.85), giving a p value of <0.0001. This indicates that NHERF-1 is significantly increased in the ovarian cancer population, relative to the breast cancer population. The ROC area for distinguishing from colon cancer was 0.83 (95% confidence interval 0.732-0.925), giving a p value of <0.0001. This indicates that NHERF-1 is also significantly increased in the ovarian cancer population, relative to the colon cancer population.

### Example 13

### Distinguishing Ovarian Cancer from Breast and Colon Cancer (all Patients Including Male Colon Cancer Patients)

In addition to subjects having ovarian cancer and normal subjects, NHERF-1 concentrations were measured in 71 breast cancer and 73 colon cancer subjects (all patients, including male colon cancer patients). The following summarizes the results obtained.

TABLE 12

| | | | SD | SE | 95% | | | 95% |
|---|---|---|---|---|---|---|---|---|
| Patient group | n | mean | (mean) | (mean) | (mean) | median | IQR | (median) |
| Ovarian | 33 | 22.1 | 36.8 | 6.4 | 9.1-35.2 | 10.4 | 16.4 | 6.5-18.8 |
| cancer | | | | | | | | |
| Breast cancer | 71 | 12.9 | 33.1 | 3.9 | 5.1-20.8 | 2.7 | 3.0 | 2.3-3.9 |
| Colon cancer | 73 | 3.0 | 4.0 | 0.5 | 2.1-4.0 | 1.5 | 2.5 | 1.1-2.0 |

Using standard ROC analysis, the ability of NHERF-1 to distinguish ovarian cancer from these other cancers was determined. The ROC area (for all patients, including normal male donors) for distinguishing from breast cancer was 0.75 (95% confidence interval 0.64-0.85), giving a p value of <0.0001. This indicates that NHERF-1 is significantly increased in the ovarian cancer population, relative to the breast cancer population. The ROC area for distinguishing from colon cancer was 0.86 (95% confidence interval 0.732-0.925), giving a p value of <0.0001. This indicates that NHERF-1 is also significantly increased in the ovarian cancer population, relative to the colon cancer population.

### Example 14

### Study Population III

Samples were purchased through a commercial vendor and were collected from cancer patients from a site in Moscow, Russia. Samples were collected according to ProteoGenex Standard Collection Procedures, which comprise collecting blood samples using a Vacutainer SST tube (Becton Dickinson #366510 or VWR #VT6510). The tubes were inverted 5 times and allowed to clot at room temperature for 30 minutes (no more than 2 hours) then centrifuged for 10 minutes at 1300-1500×G at 4° C. Serum was then removed and transferred to polypropylene tubes and spun again. Serum was then transferred to cryovials and frozen and stored at −70° to −80° C. There were a total of 48 breast cancer patients, 72 colon cancer patients, 43 lung cancer patients and 19 prostate cancer patients.

TABLE 13
Lung cancer subject characteristics:

| | | TNM | |
|---|---|---|---|
| Patient ID | Histological Diagnosis | Classification | Grade |
| 04032 | squamous cell carcinoma | T2N0M0 | G3 |
| 04033 | squamous cell carcinoma | T4N2M0 | G3 |
| 04034 | undifferentiated carcinoma | T4N2M0 | G3 |
| 04385 | squamous cell carcinoma | T1N1M0 | G1-2 |
| 04234 | squamous cell carcinoma | T3N2M0 | G1 |
| 04235 | adenocarcinoma | T3N1M0 | G3 |
| 04236 | bronchioloalveolar carcinoma | T2N1M0 | G2 |
| 04237 | undifferentiated carcinoma | T4N2M0 | G3 |
| 04238 | squamous cell carcinoma | T2N2M1 | G2 |
| 04239 | adenocarcinoma | T4N2M0 | G1-2 |
| 04242 | unknown | T2N2M1 | unknown |
| 04244 | unknown | T3N2M1 | G2 |
| 04243 | unknown | T3N3M0 | G2 |
| 04245 | SCLC | T3N3M0 | unknown |
| 04246 | adenocarcinoma | T2N2M0 | G2 |
| 04247 | squamous cell carcinoma | T4N2M0 | G2 |
| 04248 | squamous cell carcinoma | T3NxM0 | N/A |
| 04249 | squamous cell carcinoma | T2N1M0 | G2 |
| 04250 | squamous cell carcinoma | T2N0M0 | G2 |
| 04253 | bronchoalveolar carcinoma | T1N0M0 | G2 |
| 04255 | squamous cell carcinoma | T2N0M0 | G2 |
| 04257 | squamous cell carcinoma | T3NxM0 | unknown |
| 04260 | adenocarcinoma | T3N2M0 | G2 |
| 04261 | squamous cell carcinoma | T4N3M0 | G3 |
| 04262 | adenocarcinoma | T4N3M1 | G3 |
| 04263 | adenocarcinoma | T4N3M1 | G2 |
| 04265 | squamous cell carcinoma | T3N2MO | G2 |
| 04266 | unknown | T2N1M0 | G2 |
| 04267 | unknown | T2N0M0 | G2 |
| 04268 | unknown | T2N2M0 | unknown |
| 04269 | unknown | T2N0M0 | G2 |
| 04270 | adenocarcinoma | T3N2M1 | G2 |
| 04272 | squamous cell carcinoma | T3N0M0 | G2 |
| 04273 | large cell carcinoma | T2N0M0 | G3 |
| 04275 | undifferentiated carcinoma | T2NxM0 | G3 |
| 04280 | squamous cell carcinoma | T4N2M0 | G2 |
| 04281 | squamous cell carcinoma | T2NxM0 | unknown |
| 04282 | squamous cell carcinoma | T2NxM0 | unknown |
| 04284 | undifferentiated carcinoma | T3N1M0 | G3 |
| 04285 | squamous cell carcinoma | T2NxM0 | no |
| 04286 | unknown | T2NxM0 | unknown |
| 04287 | unknown | T2NxM0 | unknown |
| 04288 | unknown | T2NxM0 | unknown |

Also included were 69 normal donors collected from the same institute. Information for each donor included: age, sex, race, tumor classification and grade of cancer, as well as smoking history, personal history and family history.

### Example 15

### NHERF-1 Immunoassay III

An indirect sandwich ELISA was used to detect NHERF-1 in patient samples. Antibodies for the ELISA were developed at Biosite using phage display methods. Biotinylated anti-NHERF-1 antibody (primary antibody) diluted into assay buffer (10 mM Tris, 150 mM NaCl, 1% BSA) to 2 μg/ml was added to a 384 Neutravidin coated plates (Pierce Product #NC19658) and allowed to incubate at room temperature for 1 hour. Wells were washed with wash buffer (20 mM Borate, 150 mM NaCl, 0.2% TWEEN®-20 surface active agent (ICI Americas)) and then samples and standards were added and allowed to incubate at room temperature for 1 hour. Wells again were washed and then fluoresceinated anti-NHERF-1 antibody (secondary antibody) diluted in assay buffer to 2 μg/ml was added and allowed to incubate at room temperature for 1 hour. Wells again were washed. Anti-fluorscein antibody conjugated to alkaline phosphatase, diluted 1/2338 into assay buffer was added and allowed to incubate at room temperature for 1 hour, followed by washing. Finally, substrate (Promega ATTOPHOS®) was added and plate was read immediately. All additions were 10 μl/well unless otherwise stated.

The plates were washed 3 times between each addition and final wash was 9 times. Standards were prepared by spiking NHERF-1 into a normal serum patient pool at concentrations ranging from 50 to 0.39 ng/ml, including a neutralized 0, which is the serum pool with excess concentration of each antibody used in the ELISA. Standards were run in 4 replicates, and samples were run singly. Reading was performed using a TECAN®Spectrafluor Plus using kinetic mode reading of fluorescence over 6 read cycles (excitation filter 430 nm and emission filter 570 nm). The assay slope (RFU/seconds) was determined and each sample concentration was determined by reference to a 5 parameter log-logistic curve fit of the standard values. Reported NHERF-1 concentrations are in ng/mL.

### Example 16

### Results III

The following summarizes the results of NHERF-1 measurements in lung cancer and normal subjects (n—number of subjects; mean—mean NHERF-1 concentration; median—median NHERF-1 concentration; SD—standard deviation; SE—standard error; 95%-95% confidence interval); IQR—interquartile range.

TABLE 14

| | | | SD | SE | 95% | | | 95% |
|---|---|---|---|---|---|---|---|---|
| Patient group | n | mean | (mean) | (mean) | (mean) | median | IQR | (median) |
| Lung cancer | 43 | 7.3 | 5.4 | 0.8 | 5.6-8.9 | 6.5 | 7.0 | 3.6-8.9 |
| Normal | 69 | 3.1 | 3.4 | 0.4 | 2.3-3.9 | 2.2 | 1.5 | 1.9-2.6 |

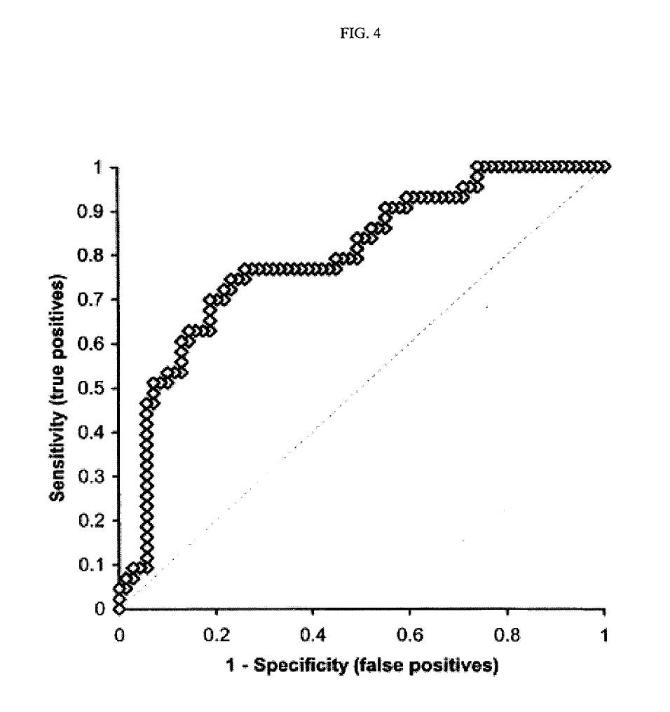

Using standard ROC analysis, the ability of NHERF-1 to distinguish lung cancer from normal was determined. The ROC area was 0.794 (95% confidence interval 0.71-0.88), giving a p value of <0.0001. This indicates that NHERF-1 is significantly increased in the lung cancer population. The ROC curve obtained is shown in FIG. 4. In addition, it was determined that it was not significantly different when comparing smokers to nonsmokers.

Odds ratios may be calculated for the combination of lung cancer and normal data. In the following example, the odds ratio is defined as the ratio of the odds of an event occurring above a selected NHERF-1 concentration, relative to the odds of it occurring below that threshold. Three thresholds were selected: the 75th percentile concentration in normal subjects, the mean concentration of the combined normal and lung cancer population, and the median concentration in that population. Table 15 summarizes the results obtained.

TABLE 15

| | | Mean of | Median of |
|---|---|---|---|
| | 75^{th} | Normal + | Normal + |
| | Percentile | Lung Cancer | Lung Cancer |
| Threshold Criteria: | of Normals | Cohorts | Cohorts |
| Threshold NHERF-1 | 2.9 | 4.7 | 2.7 |
| Concentration: | | | |
| Odds Ratio | 8.2 | 9.3 | 6.6 |
| 95% confidence interval | 3.5-19.7 | 3.7-23.4 | 2.8-15.7 |
| p value, 2 tail (OR > 1): | 2-1 × 10⁻⁶ | 2.5 × 10⁻⁶ | 2.0 × 10⁻⁵ |

These data indicate that the odds of having lung cancer are significantly increased in a subject if the NHERF-1 concentration measured in that subject exceeds any one of these threshold concentrations. Particularly striking is the fact that an individual having a concentration that exceeds the 75^{th} percentile of normal has more than an 8-fold greater probability of having lung cancer, compared to the probability when the concentration is less than the 75^{th} percentile of normal.

### Example 17

### Distinguishing Lung Cancer from Breast, Prostate and Colon Cancer I

In addition to subjects having lung cancer and normal subjects, NHERF-1 concentrations were also measured in 48 breast cancer, 19 prostate cancer, and 72 colon cancer subjects. The following summarizes the results obtained.

TABLE 16

| | | | SD | SE | 95% | | | 95% |
|---|---|---|---|---|---|---|---|---|
| Patient group | n | mean | (mean) | (mean) | (mean) | median | IQR | (median) |
| Lung cancer | 43 | 7.3 | 5.4 | 0.8 | 5.6-8.9 | 6.5 | 7.0 | 3.6-8.9 |
| Breast cancer | 48 | 5.7 | 10.6 | 1.5 | 2.6-8.8 | 2.7 | 2.6 | 2.2-3.4 |
| Colon cancer | 72 | 3.3 | 3.5 | 0.4 | 2.5-4.2 | 2.4 | 1.9 | 1.9-2.9 |
| Prostate cancer | 19 | 3.7 | 4.1 | 1.9 | 1.7-5.7 | 2.4 | 2.4 | 1.2-3.8 |

Using standard ROC analysis, the ability of NHERF-1 to distinguish lung cancer from these other cancers was determined. The ROC area for distinguishing from breast cancer was 0.70 (95% confidence interval 0.59-0.81), giving a p value of <0.0001. This indicates that NHERF-1 is significantly increased in the lung cancer population, relative to the breast cancer population. The ROC area for distinguishing from colon cancer was 0.77 (95% confidence interval 0.69-0.86), giving a p value of <0.0001. This indicates that NHERF-1 is also significantly increased in the lung cancer population, relative to the colon cancer population. And the ROC area for distinguishing from prostate cancer was 0.75 (95% confidence interval 0.62-0.89), giving a p value of <0.0001. This indicates that NHERF-1 is also significantly increased in the lung cancer population, relative to the prostate cancer population.

### Example 18

### Study Population IV

Samples were purchased through a commercial vendor and were collected from cancer patients from a site in Moscow, Russia. Samples were collected according to ProteoGenex Standard Collection Procedures, which comprise collecting blood samples using a Vacutainer SST tube (Becton Dickinson #366510 or VWR #VT6510). The tubes were inverted 5 times and allowed to clot at room temperature for 30 minutes (no more than 2 hours) then centrifuged for 10 minutes at 1300-1500×G at 4° C. Serum was then removed and transferred to polypropylene tubes and spun again. Serum was then transferred to cryovials and frozen and stored at −70° to −80° C. There were a total of 71 breast cancer patients, 73 colon cancer patients, 60 lung cancer patients and 24 prostate cancer patients.

TABLE 17
Lung cancer subject characteristics:

| | | TNM | |
|---|---|---|---|
| Patient ID | Histological Diagnosis | Classification | Grade |
| 04032 | Squamous cell carcinoma | T2N0M0 | G3 |
| 04033 | Squamous cell carcinoma | T4N2M0 | G3 |
| 04034 | undifferentiated carcinoma | T4N2M0 | G3 |
| 04385 | Squamous cell carcinoma | T1N1M0 | G1-2 |
| 04234 | Squamous cell carcinoma | T3N2M0 | G1 |
| 04235 | adenocarcinoma | T3N1M0 | G3 |
| 04236 | bronchioloalveolar carcinoma | T2N1M0 | G2 |
| 04237 | undifferentiated carcinoma | T4N2M0 | G3 |
| 04238 | Squamous cell carcinoma | T2N2M1 | G2 |
| 04239 | adenocarcinoma | T4N2M0 | G1-2 |
| 04242 | Squamous cell carcinoma | T2N2M1 | unknown |
| 04244 | Squamous cell carcinoma | T3N2M1 | G2 |
| 04243 | Squamous cell carcinoma | T3N3M0 | G2 |
| 04245 | SCLC | T3N3M0 | unknown |
| 04246 | adenocarcinoma | T2N2M0 | G2 |
| 04247 | Squamous cell carcinoma | T4N2M0 | G2 |
| 04248 | squamous cell carcinoma | T3NxM0 | unknown |
| 04249 | squamous cell carcinoma | T2N1M0 | G2 |
| 04250 | squamous cell carcinoma | T2N0M0 | G2 |
| 04253 | bronchoalveolar carcinoma | T1N0M0 | G2 |
| 04255 | squamous cell carcinoma | T2N0M0 | G2 |
| 04257 | squamous cell carcinoma | T3N0M0 | unknown |
| 04260 | adenocarcinoma | T3N2M0 | G2 |
| 04261 | squamous cell carcinoma | T4N3M0 | G3 |
| 04262 | adenocarcinoma | T4N3M1 | G3 |
| 04263 | adenocarcinoma | T4N3M1 | G2 |
| 04265 | squamous cell carcinoma | T3N2MO | G2 |
| 04266 | squamous cell carcinoma | T2N1M0 | G2 |
| 04267 | squamous cell carcinoma | T2N0M0 | G2 |
| 04268 | bronchoalveolar carcinoma | T2N2M0 | unknown |
| 04269 | adenocarcinoma | T2N0M0 | G2 |
| 04270 | adenocarcinoma | T3N2M1 | G2 |
| 04272 | squamous cell carcinoma | T3N0M0 | G2 |
| 04273 | large cell carcinoma | T2N0M0 | G3 |
| 04275 | undifferentiated carcinoma | T2N1M0 | G3 |
| 04280 | squamous cell carcinoma | T4N2M0 | G2 |
| 04281 | squamous cell carcinoma | T2N0M0 | unknown |
| 04282 | squamous cell carcinoma | T2N1M0 | unknown |
| 04284 | undifferentiated carcinoma | T3N1M0 | G3 |
| 04285 | squamous cell carcinoma | T2N0M0 | unknown |
| 04286 | squamous cell carcinoma | T2N2M0 | unknown |
| 04287 | squamous cell carcinoma | T2N0M0 | unknown |
| 04288 | squamous cell carcinoma | T2NxM0 | unknown |
| 04290 | squamous cell carcinoma | T2N2M0 | G3 |
| 04291 | squamous cell carcinoma | T1N0M0 | G3 |
| 04294 | squamous cell carcinoma | T2N2M0 | G2 |
| 04295 | large cell carcinoma | T2N1M0 | unknown |
| 04297 | squamous cell carcinoma | T2N1M0 | G2 |
| 04298 | squamous cell carcinoma | T2N0M0 | unknown |
| 04399 | squamous cell carcinoma | T2N1M0 | G2 |
| 04300 | squamous cell carcinoma | T2N0M0 | G1-2 |
| 04302 | adenocarcinoma | T2N0M0 | G2 |
| 04303 | bronchoalveolar carcinoma | T1N0M0 | G2-3 |
| 04304 | adenocarcinoma | T2N1M0 | G2 |
| 04305 | squamous cell carcinoma | T2N0M0 | G3 |
| 04306 | squamous cell carcinoma | T2N0M0 | unknown |
| 04309 | small cell carcinoma | T1N0M0 | unknown |
| 04310 | squamous cell carcinoma | T3N1M0 | G2 |
| 04311 | small cell carcinoma | T1N2M0 | unknown |
| 04312 | squamous cell carcinoma | T2N2M1 | G3 |

Information for each donor included: age, sex, race, tumor classification and grade of cancer, as well as smoking history, personal history and family history.

### Example 19

### NHERF-1 Immunoassay IV

An indirect sandwich assay using a Luminex assay platform was used to detect NHERF-1 in patient samples. Antibodies for the ELISA were developed at Biosite using phage display methods. Custom modified Luminex xMap™ magnetic beads covalently linked to an anti-NHERF-1 antibody (primary antibody) were diluted into assay buffer (50 mM Sodium Phosphate, 150 mM NaCL, 0.02% Tween20, 1% BSA) to 50,000 beads/ml. 50 μl of diluted beads were added to each well of a non-binding 96-well round bottom plate (Corning Product# 3605). Using a magnetic 96-well plate separator, the beads were pulled to the sides of the wells, washed and resuspended three times with 100 μl of assay buffer. The samples and standards were added to the beads and allowed to incubate for 1 hour at room temperature on an orbital shaker. After the beads were washed and re-suspended again, biotinylated anti-NHERF-1 antibody (secondary antibody) diluted in assay buffer to 0.05 μg/ml was added and allowed to incubate at room temperature for 1 hour on an orbital shaker. After washing and resuspension of the beads again, Streptavidin Phycoerthryin (PROzyme Phycolink Code #PJ31S) diluted to 4 μg/ml in assay buffer was added and allowed to incubate for 1 hour at room temperature on an orbital shaker. After the final wash and resuspension, the beads were passed through the flow cell of a Luminex 200 reader to measure assay signals.

Standards were prepared by spiking NHERF-1 into normal serum patient pool at concentrations ranging from 100 ng/ml to 3.13 ng/ml, including a neutralized 0, which is the serum pool with excess concentrations of each antibody used in the sandwich assay. Standards were run in 2 replicates and samples were run singly. The assay median taken from a minimum of 50-100 beads count signals was determined and each sample concentration was determined by reference to a 5 parameter log-logistic curve fit of the standard values. Reported NHERF-1 concentration are in ng/mL.

### Example 20

### Results IV

The following summarizes the results of NHERF-1 measurements in lung cancer and normal subjects (n—number of subjects; mean—mean NHERF-1 concentration; median—median NHERF-1 concentration; SD—standard deviation; SE—standard error; 95%-95% confidence interval); IQR—interquartile range.

TABLE 18

| | | | SD | SE | 95% | | | 95% |
|---|---|---|---|---|---|---|---|---|
| Patient group | n | mean | (mean) | (mean) | (mean) | median | IQR | (median) |
| Lung cancer | 60 | 9.8 | 10.8 | 1.4 | 7.0-12.6 | 6.6 | 12.2 | 3.2-11.1 |
| Normal | 69 | 2.5 | 5.1 | 0.6 | 1.3-3.8 | 1.1 | 1.2 | 0.8-1.3 |

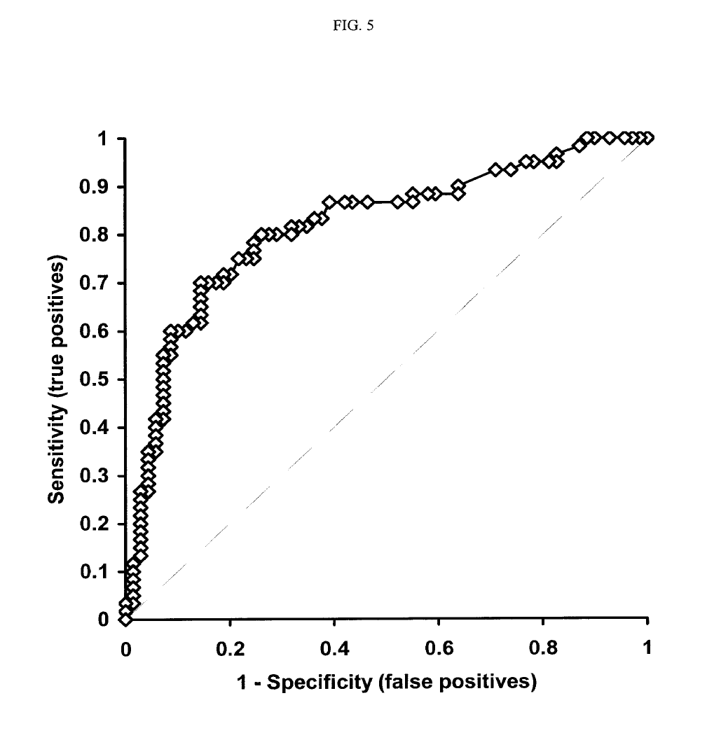

Using standard ROC analysis, the ability of NHERF-1 to distinguish lung cancer from normal was determined. The ROC area was 0.815 (95% confidence interval 0.739-0.891), giving a p value of <0.0001. This indicates that NHERF-1 is significantly increased in the lung cancer population. The ROC curve obtained is shown in FIG. 5. In addition, it was determined that it was not significantly different when comparing smokers to nonsmokers.

Odds ratios were calculated for the combination of lung cancer and normal data. Table 20 summarizes the results obtained.

TABLE 19

| | | Mean of | Median of |
|---|---|---|---|
| | | Normal + | Normal + |
| | 75^{th} Percentile | Lung Cancer | Lung Cancer |
| Threshold Criteria: | of Normals | Cohorts | Cohorts |
| Threshold NHERF-1 | 1.9 | 5.9 | 1.8 |
| Concentration: | | | |
| Odds Ratio | 11.1 | 13.7 | 10.5 |
| 95% confidence interval | 4.9-25.2 | 4.8-38.8 | 4.6-24.0 |
| p value, 2 tail (OR > 1): | 1.0 × 10⁻⁸ | 8.5 × 10⁻⁷ | 2.2 × 10⁻⁸ |

These data indicate that the odds of having lung cancer are significantly increased in a subject if the NHERF-1 concentration measured in that subject exceeds any one of these threshold concentrations. Particularly striking is the fact that an individual having a concentration that exceeds the 75^{th} percentile of normal has more than an 8-fold greater probability of having lung cancer, compared to the probability when the concentration is less than the 75^{th} percentile of normal.

### Example 21

### Distinguishing Lung Cancer from Breast, Prostate and Colon Cancer II

In addition to subjects having lung cancer and normal subjects, NHERF-1 concentrations were also measured in 71 breast cancer, 24 prostate cancer and 73 colon cancer subjects. The following table summarizes the results obtained.

TABLE 20

| | | | SD | SE | 95% | | | 95% |
|---|---|---|---|---|---|---|---|---|
| Patient group | n | mean | (mean) | (mean) | (mean) | median | IQR | (median) |
| Lung cancer | 60 | 9.8 | 10.8 | 1.4 | 7.0-12.6 | 6.6 | 12.2 | 3.2-11.1 |
| Breast cancer | 71 | 12.9 | 33.1 | 3.9 | 5.1-20.8 | 2.7 | 3.0 | 2.3-3.9 |
| Colon cancer | 73 | 3.0 | 4.0 | 0.5 | 2.1-4.0 | 1.5 | 2.5 | 1.1-2.0 |
| Prostate cancer | 24 | 8.2 | 14.1 | 2.9 | 2.3-14.2 | 2.0 | 7.5 | 0.8-6.6 |

Using standard ROC analysis, the ability of NHERF-1 to distinguish lung cancer from these other cancers was determined. The ROC area for distinguishing from breast cancer was 0.61 (95% confidence interval 0.51-0.71), giving a p value of 0.01. This indicates that NHERF-1 is significantly increased in the lung cancer population, relative to the breast cancer population. The ROC area for distinguishing from colon cancer was 0.76 (95% confidence interval 0.67-0.84), giving a p value of <0.0001. This indicates that NHERF-1 is also significantly increased in the lung cancer population, relative to the colon cancer population. And the ROC area for distinguishing from prostate cancer was 0.66 (95% confidence interval 0.51-0.78), giving a p value of 0.01. This indicates that NHERF-1 is also significantly increased in the lung cancer population, relative to the prostate cancer population.

One skilled in the art readily appreciates that the present invention is well adapted to carry out the objects and obtain the ends and advantages mentioned, as well as those inherent therein. The examples provided herein are representative of various aspects and embodiments. They are not intended as limitations on the scope of the invention.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, for example, in each instance herein any of the terms “comprising”, “consisting essentially of and “consisting of may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various aspects and embodiments, modification and variation of the concepts herein disclosed may be resorted to by those skilled in the art, and that such modifications and variations are considered to be within the scope of this invention as defined by the appended claims.

While various aspects and embodiments of the present invention have been shown and described herein, it will be obvious to those skilled in the art that such aspects and embodiments are provided by way of example only. Numerous variations, changes, and substitutions will occur to those skilled in the art without departing from the invention. It should be understood that various alternatives to the aspects and embodiments of the invention described herein may be employed in practicing the invention. It is intended that the claims define the scope of the invention and that methods and structures within the scope of these claims and their equivalents be covered thereby.

Other aspects and embodiments are set forth within the following claims.

What is claimed is:

1. A method of assigning a diagnosis to a subject being assessed for the presence of ovarian cancer, assigning a prognostic risk to a subject suffering from ovarian cancer, and/or monitoring the course of ovarian cancer treatment in a subject, the method comprising:
performing an assay that detects NHERF-1 or a marker related thereto on a sample obtained from the subject to provide an assay result; and
relating the assay result obtained to the presence or absence of ovarian cancer in the subject, to the likelihood of an outcome related to ovarian cancer in the subject, and/or to the success or failure of treatment for ovarian cancer received by the subject.

2. A method according to claim 1, wherein the assay is an immunoassay.

3. A method according to claim 1, wherein the method is a method of assigning a diagnosis to a subject being assessed for the presence of ovarian cancer, and the relating step comprises calculating an NHERF-1 concentration for the subject from the assay result and comparing the NHERF-1 concentration to a predetermined NHERF-1 threshold concentration,
wherein the subject is assigned an increased likelihood of having ovarian cancer when the NHERF-1 concentration is greater than the threshold concentration, relative to a likelihood of having ovarian cancer assigned when the NHERF-1 concentration is less than the threshold concentration.

4. A method according to claim 3, wherein the threshold concentration is obtained by a method comprising:
performing the assay on samples obtained a first group of subjects suffering from ovarian cancer, and from a second group of subjects not suffering from ovarian cancer; and
selecting a threshold concentration that distinguishes the first group from the second group with an odds ratio of at least 1.5.

5. A method according to claim 1, wherein the method is a method of assigning a prognostic risk to a subject suffering from ovarian cancer, and the relating step comprises calculating an NHERF-1 concentration for the subject from the assay result and comparing the NHERF-1 concentration to a predetermined NHERF-1 threshold concentration,
wherein the subject is assigned an increased likelihood of having a poor ovarian cancer outcome when the NHERF-1 concentration is greater than the threshold concentration, relative to a likelihood of having a poor ovarian cancer outcome assigned when the NHERF-1 concentration is less than the threshold concentration.

6. A method according to claim 5, wherein the threshold concentration is obtained by a method comprising:
performing the assay method on samples obtained a first group of subjects suffering from ovarian cancer and from a second group of subjects suffering from ovarian cancer, wherein individuals in the first group have a 5-year survival rate that is less than the second group; and
selecting a threshold concentration that distinguishes the first group from the second group with an odds ratio of at least 1.5.

7. A method according to claim 5, wherein the threshold concentration is obtained by a method comprising:
performing the assay method on a sample obtained from the subject at a time earlier than the used to provide the assay result, thereby providing an earlier assay result, and selecting a NHERF-1 concentration calculated from the earlier assay result as the threshold.

8. A method according to claim 1, wherein the method is a monitoring the course of ovarian cancer treatment in a subject, and the relating step comprises calculating an NHERF-1 concentration for the subject from the assay result and comparing the NHERF-1 concentration to a predetermined NHERF-1 threshold concentration,
wherein the subject is assigned an increased likelihood of treatment success when the NHERF-1 concentration is greater than the threshold concentration, relative to a likelihood of treatment success assigned when the NHERF-1 concentration is less than the threshold concentration.

9. A method according to claim 8, wherein the threshold concentration is obtained by a method comprising:
performing the assay method on samples obtained a first group of subjects suffering from ovarian cancer and from a second group of subjects suffering from ovarian cancer, wherein individuals in the first group have a 5-year survival rate that is less than the second group; and
selecting a threshold concentration that distinguishes the first group from the second group with an odds ratio of at least 1.5.

10. A method according to claim 8, wherein the threshold concentration is obtained by a method comprising:
performing the assay method on a sample obtained from the subject at a time earlier than the used to provide the assay result, thereby providing an earlier assay result, and selecting a NHERF-1 concentration calculated from the earlier assay result as the threshold.

11. A method according to claim 1, wherein the assay method further comprises performing one or more additional assays that detect one or more additional markers other than NHERF-1 or a marker related thereto on one or more samples obtained from the subject, thereby providing one or more additional assay results, and the relating step comprises relating the assay result and the one or more additional assay results obtained to the presence or absence of ovarian cancer in the subject, to the likelihood of an outcome related to ovarian cancer in the subject, and/or to the success or failure of treatment received by the subject.

12. A method according to claim 11, wherein the one or more additional assays detect one or more markers selected from CA 125, carcinoembryonic antigen (CEA), α-fetoprotein (AFP), human chorionic gonadotropin (β-hCG), alpha-1-antitrypsin, alpha(v) integrin, alpha(v) beta(6) Integrin, ATP7B, beta-2-microglobulin), beta III tubulin, CA54/61, CA 72-4, CA125 II, caGT (cancer-associated galactosyltransferase antigen), CASA or YKL-40, cathepsin B, CD24, CD34, c-Etsl, creatine kinase B, COX-1, EMMPRIN (extracellular matrix metalloproteinase inducer), Ep-CAM (epithelial cell adhesion molecule), Ets-1, GAT (galactosyltransferase associated with tumor), GEP (granulin-epithelin precursor), GT-II (galactosyltransferase isozyme II), human epididymis protein 4 (HE4), HER-2, hK8 (human kallikrein 8), hK10 (10 (human kallikrein 10), hK13 (human kallikrein 13), HLA-G, HNF-1β IAP (immunosuppressive acidic protein), IGFBP-2, KLK9 (kallikrein gene 9), M-CAM (melanoma cell adhesion molecule), M-CSF (macrophage colony-stimulating factor), mesothelin, MMP-2 (matrix metalloproteinase-2), nm23-H1, osteopontin, p53, P-III-P (type III procollagen peptide), P-glycoprotein, PP-4 (mlacental protein 4), mrogesterone, progesterone receptor (PR), prostasin, PUMP-1, sialyl SSEA-1 antigen, SM047, STN antigen (serum sialyl Tn antigen), TAG-72, thymidine phosphorylase (TP), TNF Receptor p75, topoisomerase II, tPA (tissue plasminogen activator), VSGP/F-spondin, WT-1, YB-1 (Y box-binding protein-1), P-gp (P-glycoprotein), YKL-40 and podocalyxin-like protein 1.

13. A method according to claim 1, wherein the assay method further comprises performing one or more imaging studies on the subject, and the relating step comprises relating the assay result and the results obtained from the one or more imaging studies to the presence or absence of ovarian cancer in the subject, to the likelihood of an outcome related to ovarian cancer in the subject, and/or to the success or failure of treatment received by the subject.

14. A method according to claim 13, wherein the one or more imaging studies are selected from transvaginal ultrasonography studies, computed tomography (CT) studies, magnetic resonance imaging (MRI) studies, and transvaginal color flow Doppler studies.

15. A method according to claim 1, wherein the sample is from a human.

16. A method according to claim 1, wherein the sample is selected from blood, serum, and plasma.

17. A method according to claim 1, wherein the assay is configured to detect NHERF-1 having the sequence of SEQ ED NO: 1.

18. A method according to claim 17, wherein the assay also detects one or more immunologically detectable fragments of NHERF-1 having the sequence of SEQ ID NO: 1, the fragment(s) comprising 8 or more contiguous residues thereof.

19. A method of assigning a diagnosis to a subject being assessed for the presence of ovarian cancer, assigning a prognostic risk to a subject suffering from ovarian cancer, and/or monitoring the course of ovarian cancer treatment in a subject, the method comprising:
performing an assay that detects one or more markers of a NHERF-1-containing complex on a sample obtained from the subject to provide an assay result; and
relating the assay result obtained to the presence or absence of ovarian cancer in the subject, to the likelihood of an outcome related to ovarian cancer in the subject, and/or to the success or failure of treatment for ovarian cancer received by the subject.

20. A method according to claim 19, wherein the NHERF-1-containing complex comprises NHERF-1 and one or more species selected from podocalyxin-like protein 1, EZR (ezrin), RDX (radixin), MSN (moesin), PDGFRA (platelet-derived growth factor receptor, alpha polypeptide), PDGFRB (platelet-derived growth factor receptor, beta polypeptide), ADRB2 (adrenergic, beta 2), NOS2 (nitric oxide synthase 2), CFTR (cystic fibrosis transmembrane conductance regulator), ARHGAP17 (Rho GTPase activating protein 17), EPI64 (TBC1 domain family, member 10A), GNB2L1 (guanine nucleotide binding protein, beta polypeptide 2-like 1), OPRK1 (opioid receptor, kappa 1), GNAQ (guanine nucleotide binding protein, q polypeptide), CTNNB1 (catenin (cadherin-associated protein), beta 1), PLCB3 (phospholipase C, beta 3), PDZK1 (PDZ domain containing 1), PAG1 (phosphoprotein associated with glycosphingolipid microdomains 1), SLC4A7 (solute carrier family 4, sodium bicarbonate cotransporter, member 7), ATP6V1B1 (ATPase), HTR4 (5 hydroxytryptamine (serotonin) receptor 4), CLCN3 (chloride channel protein 3) and SLC9A3R2 (sodium-hydrogen exchanger regulatory factor 2).

21. A method according to claim 19, wherein the NHERF-1-containing complex comprises NHERF-1 and podocalyxin-like protein 1.

22. A method of assigning a diagnosis to a subject being assessed for the presence of lung cancer, assigning a prognostic risk to a subject suffering from lung cancer, and/or monitoring the course of lung cancer treatment in a subject, the method comprising:
performing an assay that detects NHERF-1 or a marker related thereto on a sample obtained from the subject to provide an assay result; and
relating the assay result obtained to the presence or absence of lung cancer in the subject, to the likelihood of an outcome related to lung cancer in the subject, and/or to the success or failure of treatment for lung cancer received by the subject.

23. A method according to claim 22, wherein the assay is an immunoassay.

24. A method according to claim 22, wherein the method is a method of assigning a diagnosis to a subject being assessed for the presence of lung cancer, and the relating step comprises calculating an NHERF-1 concentration for the subject from the assay result and comparing the NHERF-1 concentration to a predetermined NHERF-1 threshold concentration,
wherein the subject is assigned an increased likelihood of having lung cancer when the NHERF-1 concentration is greater than the threshold concentration, relative to a likelihood of having lung cancer assigned when the NHERF-1 concentration is less than the threshold concentration.

25. A method according to claim 24, wherein the NHERF-1 threshold concentration is obtained by a method comprising:
performing the assay on samples obtained a first group of subjects suffering from lung cancer, and from a second group of subjects not suffering from lung cancer; and
selecting a threshold concentration that distinguishes the first group from the second group with an odds ratio of at least 1.5.

26. A method according to claim 22, wherein the method is a method of assigning a prognostic risk to a subject suffering from lung cancer, and the relating step comprises calculating an NHERF-1 concentration for the subject from the assay result and comparing the NHERF-1 concentration to a predetermined NHERF-1 threshold concentration,
wherein the subject is assigned an increased likelihood of having a poor lung cancer outcome when the NHERF-1 concentration is greater than the threshold concentration, relative to a likelihood of having a poor lung cancer outcome assigned when the NHERF-1 concentration is less than the threshold concentration.

27. A method according to claim 26, wherein the threshold concentration is obtained by a method comprising:
performing the assay method on samples obtained a first group of subjects suffering from lung cancer and from a second group of subjects suffering from lung cancer, wherein individuals in the first group have a 5-year survival rate that is less than the second group; and
selecting a threshold concentration that distinguishes the first group from the second group with an odds ratio of at least 1.5.

28. A method according to claim 26, wherein the threshold concentration is obtained by a method comprising:
performing the assay method on a sample obtained from the subject at a time earlier than the used to provide the assay result, thereby providing an earlier assay result, and selecting a NHERF-1 concentration calculated from the earlier assay result as the threshold.

29. A method according to claim 22, wherein the method is a monitoring the course of lung cancer treatment in a subject, and the relating step comprises calculating an NHERF-1 concentration for the subject from the assay result and comparing the NHERF-1 concentration to a predetermined NHERF-1 threshold concentration,
wherein the subject is assigned an increased likelihood of treatment success when the NHERF-1 concentration is greater than the threshold concentration, relative to a likelihood of treatment success assigned when the NHERF-1 concentration is less than the threshold concentration.

30. A method according to claim 29, wherein the NHERF-1 threshold concentration is obtained by a method comprising:
performing the assay method on samples obtained a first group of subjects suffering from lung cancer and from a second group of subjects suffering from lung cancer, wherein individuals in the first group have a 5-year survival rate that is less than the second group; and
selecting a threshold concentration that distinguishes the first group from the second group with an odds ratio of at least 1.5.

31. A method according to claim 29, wherein the NHERF-1 threshold concentration is obtained by a method comprising:
performing the assay method on a sample obtained from the subject at a time earlier than the used to provide the assay result, thereby providing an earlier assay result, and selecting a NHERF-1 concentration calculated from the earlier assay result as the threshold.

32. A method according to claim 22, wherein the assay method further comprises performing one or more additional assays that detect one or more additional markers other than NHERF-1 or a marker related thereto on one or more samples obtained from the subject, thereby providing one or more additional assay results, and the relating step comprises relating the assay result and the one or more additional assay results obtained to the presence or absence of lung cancer in the subject, to the likelihood of an outcome related to lung cancer in the subject, and/or to the success or failure of treatment received by the subject.

33. A method according to claim 32, wherein the one or more additional assays detect one or more markers selected from CA 125, carcinoembryonic antigen (CEA), neuron-specific enolase (NSE), cytokeratin 19 fragments (CYFRA 21-1), HER2-neu, progastrin-releasing peptide (ProGRP), squamous cancer cell antigen (SCCA), tissue polypeptide antigen (TPA), tissue polypeptide specific-antigen (TPS), tumor M2 pyruvate kinase (TU M2-PK), ferritin, soluble interleukin-2 receptor (sIL-2r), creatine kinase-BB (CK-BB), glycosyl transferase, bombesin/gastrin releasing peptide, adrenocorticotropin (ACTH), antidiuretic hormone (ADH), calcitonin, insulin-like growth factor-I (IGF-I), osteopontin, human epididymis protein 4 (HE4), insulin-like growth factor-II (IGF-II) and podocalyxin-like protein 1.

34. A method according to claim 22, wherein the assay method further comprises performing one or more imaging studies on the subject, and the relating step comprises relating the assay result and the results obtained from the one or more imaging studies to the presence or absence of lung cancer in the subject, to the likelihood of an outcome related to lung cancer in the subject, and/or to the success or failure of treatment received by the subject.

35. A method according to claim 34, wherein the one or more imaging studies are selected from conventional X-ray, tomography (CT), magnetic resonance imaging (MRI), and positron emission tomography (PET) imaging studies.

36. A method according to claim 22, wherein the sample is from a human.

37. A method according to claim 22, wherein the sample is selected from blood, serum, and plasma.

38. A method according to claim 22, wherein the assay is configured to detect NHERF-1 having the sequence of SEQ ID NO: 1.

39. A method according to claim 38, wherein the assay also detects one or more immunologically detectable fragments of NHERF-1 having the sequence of SEQ ID NO: 1, the fragment(s) comprising 8 or more contiguous residues thereof.

40. A method of assigning a diagnosis to a subject being assessed for the presence of lung cancer, assigning a prognostic risk to a subject suffering from lung cancer, and/or monitoring the course of lung cancer treatment in a subject, the method comprising:
performing an assay that detects one or more markers of a NHERF-1-containing complex on a sample obtained from the subject to provide an assay result; and
relating the assay result obtained to the presence or absence of lung cancer in the subject, to the likelihood of an outcome related to lung cancer in the subject, and/or to the success or failure of treatment for lung cancer received by the subject.

41. A method according to claim 40, wherein the NHERF-1-containing complex comprises NHERF'-1 and one or more species selected from podocalyxin-like protein 1, EZR (ezrin), RDX (radixin), MSN (moesin), PDGFRA (platelet-derived growth factor receptor, alpha polypeptide), PDGFRB (platelet-derived growth factor receptor, beta polypeptide), ADRB2 (adrenergic, beta 2), NOS2 (nitric oxide synthase 2), CFTR (cystic fibrosis transmembrane conductance regulator), ARHGAP17 (Rho GTPase activating protein 17), EPI64 (TBC1 domain family, member 10A), GNB2L1 (guanine nucleotide binding protein, beta polypeptide 2-like 1), OPRK1 (opioid receptor, kappa 1), GNAQ (guanine nucleotide binding protein, q polypeptide), CTNNB1 (catenin (cadherin-associated protein), beta 1), PLCB3 (phospholipase C, beta 3), PDZK1 (PDZ domain containing 1), PAG1 (phosphoprotein associated with glycosphingolipid microdomains 1), SLC4A7 (solute carrier family 4, sodium bicarbonate cotransporter, member 7), ATP6V1B1 (ATPase), HTR4 (5 hydroxytryptamine (serotonin) receptor 4), CLCN3 (chloride channel protein 3) and SLC9A3R2 (sodium-hydrogen exchanger regulatory factor 2).

42. A method according to claim 40, wherein the NHERF-1-containing complex comprises NHERF-1 and podocalyxin-like protein 1.
